(19) United States
(12) Patent Application Publication
(10) Pub. No.: US 2024/0368072 A1
(43) Pub. Date: Nov. 7, 2024

(54) METHOD FOR PREPARATION OF N-ACETYL CYSTEINE AMIDE OR DI- N-ACETYL CYSTEINE AMIDE AND DERIVATIVES

(71) Applicant: NACUITY PHARMACEUTICALS, INC., Fort Worth, TX (US)

(72) Inventors: G. Michael Wall, Fort Worth, TX (US); Gerald D. Cagle, Fort Worth, TX (US)

(21) Appl. No.: 18/770,850

(22) Filed: Jul. 12, 2024

Related U.S. Application Data
(63) Continuation-in-part of application No. 18/678,550, filed on May 30, 2024.
Application No. 18/678,550 is a continuation-in-part of application No. 18/094,459, filed on Jan. 9, 2023.
Application No. 18/094,459 is a continuation-in-part of application No. 17/366,913, filed on Jul. 2, 2021, now Pat. No. 11,548,851.
Application No. 17/366,913 is a continuation-in-part of application No. 16/818,416, filed on Mar. 13, 2020, now Pat. No. 11,091,433.
Application No. 16/818,416 is a continuation-in-part of application No. 16/137,262, filed on Sep. 20, 2018, now Pat. No. 10,590,073.
(60) Provisional application No. 62/561,129, filed on Sep. 20, 2017.

(51) Int. Cl. C07C 231/02; A61K 31/16

(52) U.S. Cl. CPC C07C 231/02; A61K 31/16

(57) ABSTRACT

Provided herein are compositions and methods of providing an effective amount of an N-acetylcysteine amide (NACA) or diNACA sufficient to increase the concentration of NACA or diNACA in the affected tissue to biochemically reduce oxidative stress-related damage to tissue; utilizing NACA or diNACA that comprises at least one of impurities B1 or B2, which are indicative of NACA or diNACA manufactured by a process comprising: contacting cystine with methanol and a chlorinating reagent to form an organic solution containing cystine dimethylester dihydrochloride and optionally isolating and drying the cystine dimethylester dihydrochloride; combining the dried or undried cystine dimethylester dihydrochloride with triethylamine, acetic anhydride, and acetonitrile to form di-N-acetylcystine dimethylester; mixing the di-N-acetylcystine dimethylester with ammonium hydroxide to form di-N-acetylcystine amide (diNACA or NPI-002); and reducing the di-N-acetylcystine amide to N-acetylcysteine, NACA or NPI-001 with a reducing agent, an organic solvent, and a base.

### CROSS-REFERENCE TO RELATED APPLICATIONS

This application claims priority to and is a Continuation-in-Part of U.S. Ser. No. 18/678,550, filed May 30, 2024, entitled “Method for Preparation of N-Acetyl Cysteine Amide or DI-N-Acetyl Cystineamide and Derivatives”, which is a Continuation-in-Part of U.S. Ser. No. 18/094,459, filed Jan. 9, 2023, entitled “Method for Preparation of N-Acetyl Cysteine Amide or DI-N-Acetyl Cystineamide and Derivatives”, which is a Continuation-in-Part of U.S. Ser. No. 17/366,913, filed Jul. 2, 2021, entitled “Method for Preparation of N-Acetyl Cysteine Amide and Derivatives Thereof”, now U.S. Pat. No. 11,548,851, which is a Continuation-in-Part of U.S. Ser. No. 16/818,416, filed Mar. 13, 2020, entitled “Method for Preparation of N-Acetyl Cysteine Amide and Derivatives Thereof”, now U.S. Pat. No. 11,091,433, which is a Continuation-in-Part of U.S. Ser. No. 16/137,262, filed Sep. 20, 2018, entitled “Method for Preparation of N-Acetyl Cysteine Amide and Derivatives”, now U.S. Pat. No. 10,590,073, which claims priority to U.S. Provisional Application Ser. No. 62/561,129, filed Sep. 20, 2017, entitled “Method for Preparation of N-Acetyl Cysteine Amide and Derivatives”, the entire contents of each of which are incorporated herein by reference.

### STATEMENT OF FEDERALLY FUNDED RESEARCH

None.

### TECHNICAL FIELD OF THE INVENTION

The present invention relates in general to the field of N-acetylcysteine amide [NACA, NPI-001] and/or di-N-Acetylcysteine Amide (diNACA) containing pharmaceutical product.

### BACKGROUND OF THE INVENTION

Without limiting the scope of the invention, its background is described in connection with N-acetylcysteine amide (NACA) and di-N-Acetylcysteine Amide (diNACA).

NACA synthesized according to U.S. Pat. No. 9,763,902B2 was found to contain “˜4-5% of disulfide of N-aceyl-L-cysteine amide (di-NACA)” or diNACA. For Nacuity's global development of NPI-001 as a pharmaceutical product, 4-5% diNACA content may be considered an excessive level of impurity generally not acceptable to regulatory agencies. According to U.S. Pat. No. 9,763,902B2, flash chromatography must be used to greatly decrease the diNACA content of NACA. N-acetylcysteine amide, NACA or “NPI-001” drug substance or drug product manufactured according to the process of U.S. Pat. No. 10,590,073 yields NACA of greater purity, i.e., “purified NACA,” purified N-Acetylcysteine amide” or “purified NPI-001,” without the need for chromatographic purification, compared to U.S. Pat. No. 9,763,902B2.

NACA/NPI-001 is an active pharmaceutical ingredient (API) that is being developed for the treatment of diseases and conditions involving oxidative stress. Therefore, NPI-001 may eventually comprise a valuable product. Hence, the development of anticounterfeit measures are of interest for NACA/NPI-001-containing products manufactured by the process of U.S. patent Ser. No. 10/590,073, including isolated and purified NACA/NPI-001 API or isolated and purified NACA/NPI-001 Tablets.

### SUMMARY OF THE INVENTION

As embodied and broadly described herein, an aspect of the present disclosure relates to a drug substance used in a method for treating a disease or condition in an animal or human in need thereof, the method comprising: identifying that a subject has a disease or condition caused by oxidative stress; providing an effective amount of an N-acetylcysteine amide (NACA) sufficient to increase a concentration of NACA; and treating the disease or condition with NACA that comprises at least one of impurities B1 or B2, which are indicative of NACA manufactured by a process comprising: contacting cystine with methanol and a chlorinating reagent to form an organic solution containing cystine dimethylester dihydrochloride and optionally isolating and drying the cystine dimethylester dihydrochloride; combining the dried or undried cystine dimethylester dihydrochloride with triethylamine, acetic anhydride, and acetonitrile to form di-N-acetylcystine dimethylester; mixing the di-N-acetylcystine dimethylester with ammonium hydroxide to form di-N-acetylcystine amide; and reducing the di-N-acetylcystine amide to NACA with a reducing agent, an organic solvent, and a base. In one aspect, the NACA comprises impurities B1 and B2 at any detectable concentration as determined by HPLC analysis. In another aspect, the NACA comprises at least one of: impurities B1 and B2 at levels greater than zero but B1 less than 0.91% peak area percents (PA %) and B2 less than 2.27 PA %, as determined by HPLC peak area analysis; impurities B1 and B2 at levels greater than zero but B1 less than about 2 PA % and B2 less than about 4 PA %, as determined by HPLC peak area analysis; impurities B1 and B2 at levels greater than zero but B1 less than 5% and B2 less than 7 PA %, as determined by HPLC peak area analysis; or impurities B1 at about 0.05 PA % to 0.5 PA % and B2 at about 1 to 2 PA %, as anticounterfeit markers. In another aspect, the NACA comprises impurities B1 and B2 at levels that have been toxicologically qualified and serve as markers for NACA. In another aspect, the NACA is provided orally, peritoneally, intravenously, dermally, bucally, sublingually, topically, topical ocularly, intraocularly, intravitreally, transmucosally, intradermally, subcutaneously, or pulmonarily. In another aspect, the NACA is administered in the form of a gel, ointment, liniment, lotion, capsule, cream, implant, minitablet, pill, powder, suspension, tablet, emulsion or a suppository. In another aspect, the disease is retinitis pigmentosa, retinitis pigmentosa associated with Usher syndrome, age-related macular degeneration, cystinosis, corneal endothelial loss, amyotrophic lateral sclerosis (ALS), Alzheimer's disease, Parkinson's disease, Huntington's disease, a liposomal storage disease, a gain of function mutation in ACOX1, or Hereditary cystatin C amyloid angiopathy (HCCAA). In another aspect, the condition is traumatic brain injury, cataract formation, binge eating, methamphetamine abuse, alcohol abuse, or acetaminophen overdose. In another aspect, the NACA is administered topically in the form of a gel, an ointment, a liniment, a lotion, a cream, a pill, a powder, a solution, a suspension, an emulsion, an implant, a sublingual formulation or a suppository. In another aspect, the NACA is administered topically in the form of a solution that is formed by mixing lyophilized NACA with diluent prior to administration. In another aspect, the NACA is administered by an intradermal, intramuscular, intraocular, intravitreal or subcutaneous injection. In another aspect, a dose of NACA is between 1 and 10 mg/day, between 10 and 200 mg/day, between 60 and 80 mg/day, between 250 and 500 mg/day, between 501 and 1,500 mg/day, or between 1,501 and 4,000 mg/day. In another aspect, the NACA is dosed daily for over several days, 1 week, 1 month, 6 months, 1 year or 5 years or longer. In another aspect, the NACA is purified as a result of crystallization, needing no further purification by at least one of: column chromatography, paper chromatography, thin layer chromatography, high performance liquid chromatography, fast liquid chromatography, supercritical fluid chromatography, affinity chromatography, reversed phase chromatography, two dimensional chromatography, counter current chromatography, or flash chromatography. In another aspect, the NACA contains less than 2 PA % diNACA or less than 4 PA % diNACA.

As embodied and broadly described herein, an aspect of the present disclosure relates to a pharmaceutical composition used for treating diseases and conditions in an animal or human comprising: an N-acetylcysteine amide (NACA) that comprises impurities B1 and B2 that are indicative of NACA manufactured by: contacting cystine with methanol and a chlorinating reagent to form an organic solution containing cystine dimethylester dihydrochloride and optionally isolating and drying the cystine dimethylester dihydrochloride; combining the dried or undried cystine dimethylester dihydrochloride with triethylamine, acetic anhydride, and acetonitrile to form di-N-acetylcystine dimethylester; mixing the di-N-acetylcystine dimethylester with ammonium hydroxide to form di-N-acetylcystine amide; and reducing the di-N-acetylcystine amide to NACA with a reducing agent, an organic solvent, and a base; and a pharmaceutically acceptable carrier. In one aspect, the NACA is a tablet is dosed once daily, dosed twice daily, or dosed more than twice daily. In another aspect, the NACA contains impurities B1 and B2 at any detectable concentration as determined by HPLC analysis. In another aspect, the NACA is not purified by chromatography.

As embodied and broadly described herein, an aspect of the present disclosure relates to a chemical mixture of N-acetylcysteine amide (NACA, NPI-001) comprising the following chemical structures:

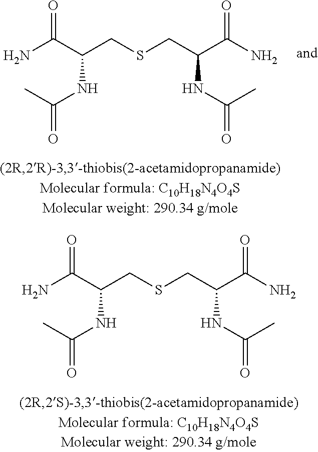 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]

wherein the NACA contains total unspecified impurities of less than 1.55 peak area percents (PA %) based on HPLC peak area. In another aspect, the total unspecified impurities at about ≤0.24 PA %, less than about 0.5 PA %, less than about 1 PA %, less than about 1.55 PA %. In another aspect, the mixture contains less than 2 PA % diNACA, or less than 4 PA % diNACA. In another aspect, the mixture is a purified NACA.

As embodied and broadly described herein, an aspect of the present disclosure relates to a chemical moieties with the following chemical structures:

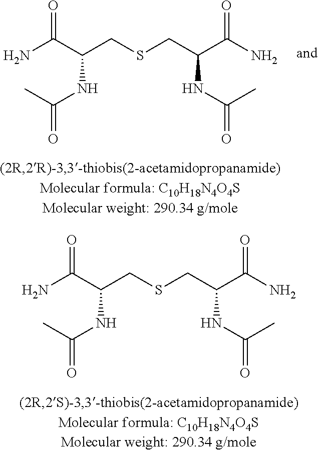 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]

As embodied and broadly described herein, an aspect of the present disclosure relates to a NACA drug substance or drug product prepared by a method comprising: contacting cystine with methanol and a chlorinating reagent to form an organic solution containing cystine dimethylester dihydrochloride and optionally isolating and drying the cystine dimethylester dihydrochloride; combining the dried or undried cystine dimethylester dihydrochloride with triethylamine, acetic anhydride, and acetonitrile to form di-N-acetylcystine dimethylester; mixing the di-N-acetylcystine dimethylester with ammonium hydroxide to form di-N-acetylcystine amide; and reducing the di-N-acetylcystine amide to NACA with a reducing agent, an organic solvent, and a base, wherein the drug substance or drug product comprises at least one of: impurity B1 as an identifier, indicator, marker, or anticounterfeit measure; or impurity B2 as an identifier, indicator, marker, or anticounterfeit measure. In one aspect, the drug substance or drug product comprises at least one of: about 0.05 to 0.5% to 1% impurity B1; or less than 5% impurity B1; about 1 to 1.5% to 2% impurity B2; or less than 5% impurity B2. In another aspect, the presence of at least one of impurity B1 or impurity B2 comprises an anticounterfeit marker, identifier or indicator of the synthetic process.

NPI-001 drug substance or drug product manufactured by the process of U.S. Pat. No. 10,590,073 yields NACA, without the need for chromatographic purification, compared to U.S. Pat. No. 9,763,902B2 which does require chromatographic purification.

In accordance with an embodiment, the present invention provides anticounterfeit measures for purified NPI-001-containing pharmaceutical product. The unique Impurity profile of NPI-001 manufactured by the process of U.S. patent Ser. No. 10/590,073, i.e., the presence of impurities B1 and B2, serves as an identifier, indicator, marker, reporter and anticounterfeit measure that purified NACA, NPI-001 API or purified NPI-001 formulation (including Tablets) were manufactured according to the process of U.S. patent Ser. No. 10/590,073. For illustrative purposes, the formulation utilized in this document is that of purified NPI-001 Tablets but tablets are used as an example. The formulation could also be in the form of a gel, ointment, liniment, lotion, capsule, cream, implant, minitablet, pill, powder, suspension, tablet, emulsion, suppository, or any other type of formulation.

### BRIEF DESCRIPTION OF THE FIGURES

For a more complete understanding of the features and advantages of the present disclosure, reference is now made to the detailed description of the disclosure along with the accompanying figures and in which:

FIG. 1 shows a preparative scale HPLC chromatogram of Step 3A filtrate (as per process described in U.S. patent Ser. No. 10/590,073). Impurities B1 and B2 are represented as Fraction 1 (2.6 minutes) and Fraction 2 (2.9 minutes). However, it is unknown which peak corresponds to which diastereomer.

FIG. 2 shows an analytical HPLC chromatogram of residue from preparative fraction 1.

FIG. 3 shows an analytical HPLC chromatogram of residue from preparative fraction 2.

FIG. 4 shows full scan ESI+ spectrum of residue from Impurity B1.

FIG. 5 shows ESI+ MS/MS of 313.1 spectrum of Impurity B1.

FIG. 6 shows ESI+ MS/MS of 291.1 spectrum of Impurity B1.

FIG. 7 shows Full scan ESI+ spectrum of enriched Impurity B2.

FIG. 8 shows ESI+ MS/MS of 313.1 spectrum of Impurity B2.

FIG. 9 shows ¹H-NMR spectrum of Impurity B1.

FIG. 10 shows ¹³C-NMR spectrum of Impurity B1.

FIG. 11 shows COSY NMR spectrum of Impurity B1.

FIG. 12 shows HMBC NMR spectrum of Impurity B1.

FIG. 13 shows HSQC NMR spectrum of Impurity B1.

FIG. 14 shows NOESY NMR spectrum of Impurity B1.

FIG. 15 shows TOCSY NMR spectrum of Impurity B1.

FIG. 16 shows ¹H-NMR spectrum of Impurity B2.

FIG. 17 shows ¹³C-NMR spectrum of Impurity B2.

FIG. 18 shows COSY NMR spectrum of Impurity B2.

FIG. 19 shows HMBC NMR spectrum of Impurity B2.

FIG. 20 shows HSQC NMR spectrum of Impurity B2.

FIG. 21 shows NOESY NMR spectrum of Impurity B2.

FIG. 22 shows HPLC chromatogram of impurities B1 and B2 in NPI-001 Tablets.

FIG. 23 shows HPLC chromatogram of Placebo showing neither Impurity B1 nor B2, and no NPI-001.

### DETAILED DESCRIPTION OF THE INVENTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

Without limiting the scope of the invention, the unique Impurity profile of N-acetylcysteine amide (NPI-001), i.e., the presence of impurities B1 and B2, serves as an identifier, indicator, marker and anticounterfeit measure that isolated and purified N-acetylcysteine amide (NPI-001) API or Tablets were manufactured according to the process of U.S. Pat. No. 10,590,073, to which this application claims priority. The absence of impurities B1 and B2 indicates a counterfeit scenario.

A producer or reseller of pharmaceutical products, for example a manufacturer, supplier, wholesaler, distributor, repackager, or retailer, especially for, but not limited to, high-value items, faces counterfeiting of the item. Counterfeiting includes the substitution, dilution, addition or omission of ingredients or components of the item compared to its intended product specification, as well as misrepresentation or diversion of the packaged item from its intended course of sale. This leads to loss of potential revenue as counterfeit items are sold in the place of the real item. Also, there can be health or product-related damages, e.g., the counterfeit can perform differently or not at all as compared to the isolated and purified item.

The unique impurity profile of N-acetylcysteine amide (NACA, NPI-001), i.e., the presence of innocuous synthetic by-product impurities B1 and B2, serves as an identifier, indicator, marker, reporter and anticounterfeit measure that dictates NPI-001 (NACA, N-acetylcysteine amide) active pharmaceutical ingredient (API) or NPI-001 formulation (including Tablets) were manufactured according to the patented process of U.S. Pat. No. 10,590,073. NPI-001 API or NPI-001 formulation (including Tablets) manufacturedby the patented process of U.S. Pat. No. 10,590,073 are hereby referred to as “purified” NPI-001 API or “purified” NPI-001 formulation (including Tablets). The absence of impurities B1 and B2 indicates a counterfeit product.

N-Acetylcysteine amide may be synthesized by other methods. One such method involves esterification and amidation of N-acetylcysteine (NAC) as described in U.S. Pat. No. 9,763,902B2. NAC is first esterified with methanol to yield NAC-methyl ester followed by amidation with ammonia to yield NACA which was further purified by washing with ethanol (EtOH)/heptane and chromatography with methanol/dichloromethane. Unfortunately, a significant amount of the oxidation by product, diNACA (˜4-5%), was formed using this process. This process is also not suitably scalable as it generally requires undesirable solvents and chromatographic purification of the final product. Hence, the process of U.S. Pat. No. 10,590,073 was developed as an improvement to afford large-scale manufacture of NACA, NPI-001, under the U.S. Food and Drug Administration's (FDA's) Good Manufacturing Practices (GMP), herein referred to as NPI-001 or NACA or N-acetylcysteine amide.

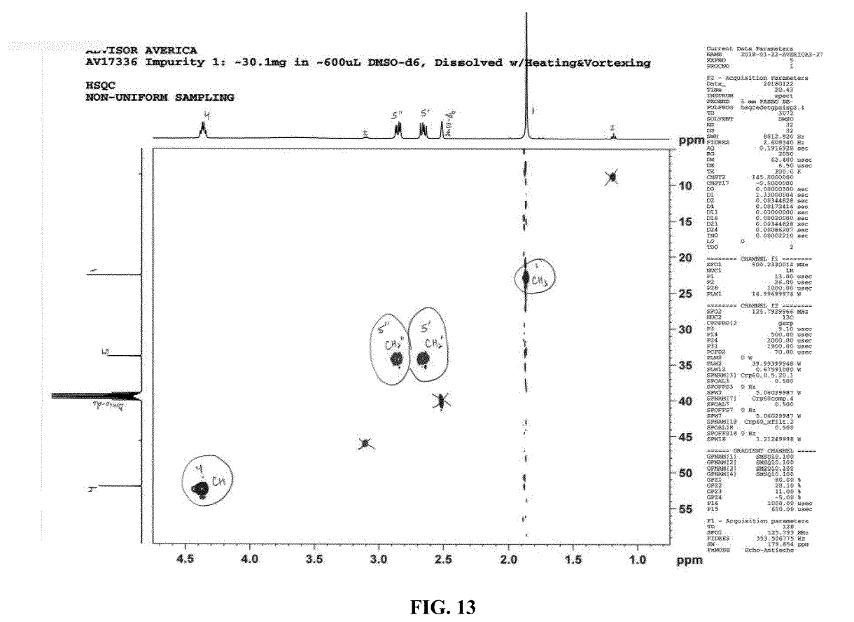

U.S. Pat. No. 10,590,073 describes a synthetic process for large-scale GMP manufacture of N-acetylcysteine amide (NPI-001) API and NPI-001 Tablets. N-acetylcysteine amide (NPI-001) API or NPI-001 Tablets manufactured according to the process of U.S. Pat. No. 10,590,073 are referred to as isolated and purified, relevant portions incorporated herein by reference. Table 7 in U.S. Pat. No. 10,590,073 describes HPLC Method II for the analysis of Impurities B1 and B2. FIG. 13 in U.S. Pat. No. 10,590,073 shows an exemplar chromatogram of impurities B1 and B2 in N-acetylcysteine amide (NACA) using HPLC Method II. NACA (NPI-001) API was manufactured/synthesized according to the process of U.S. Pat. No. 10,590,073.

As used herein, the term “purified NACA” refers to the result of the synthesis described herein, wherein the final product comprises 0.05% to 0.5% peak area percents (PA %) to 1 PA % impurity B1; or less than 5 PA % impurity B1; about 0.05 to 0.5%, or 0.5% to 1.5 PA % to 2 PA % impurity B2; or less than 5 PA % impurity B2, and/or less than 2 PA % diNACA, or less than 4 PA % diNACA. The NACA produced hereby is sufficiently pure to provide to a subject without further purification, e.g., purification by chromatography. The purity value can be determined using peak area percent of total peak area using, e.g., high performance liquid chromatography (HPLC).

As used herein, the term “purified diNACA” refers to the result of the synthesis described herein, wherein the final product comprises 0.05% to 0.5% peak area percents (PA %) to 1 PA % impurity B1; or less than 5 PA % impurity B1; about 0.05 to 0.5%, or 0.5% to 1.5 PA % to 2 PA % impurity B2; or less than 5 PA % impurity B2. The diNACA produced hereby is sufficiently pure to provide to a subject without further purification, e.g., purification by chromatography. The purity value can be determined using peak area percent of total peak area using, e.g., HPLC.

As used herein, the percent of the impurities is referred to as peak area percents (PA %) which correlates to weight area percents (WA %). The peak area percents (PA %) is the amount of the peak as calculated versus a total peak area. The PA % can also be correlated to a mole %. The total peak area can be corrected by subtracting the area values of a blank peak and a solvent peak. Unless otherwise indicated to the contrary, a percentage amount of any individual impurities (known/unknown), or total impurities reported herein in the formulations are determined by a peak area percent method using HPLC compared to a reference standard of the parent compound (either NACA or diNACA, whichever the case may be).

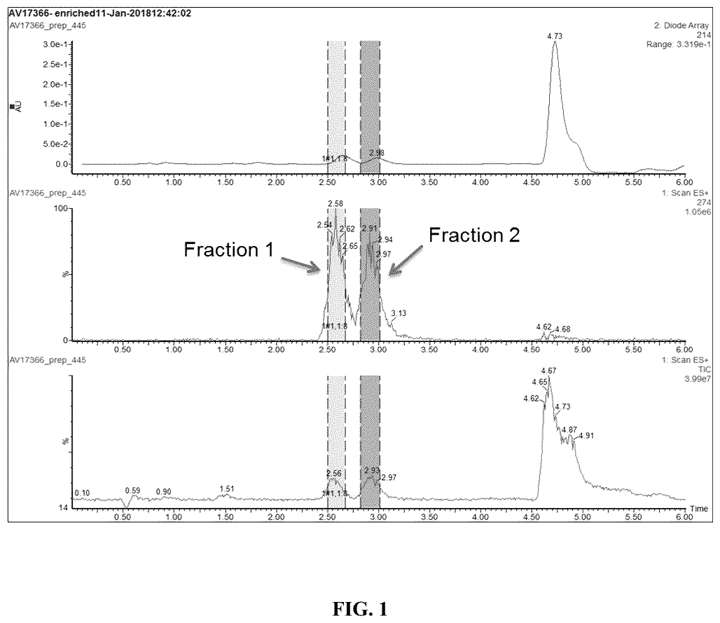

FIG. 1 shows a preparative scale HPLC chromatogram of Step 3A filtrate (as per process described in U.S. Pat. No. 10,590,073). Impurities B1 and B2 are represented as Fraction 1 (2.6 minutes) and Fraction 2 (2.9 minutes). However, it is unknown which peak corresponds to which diastereomer.

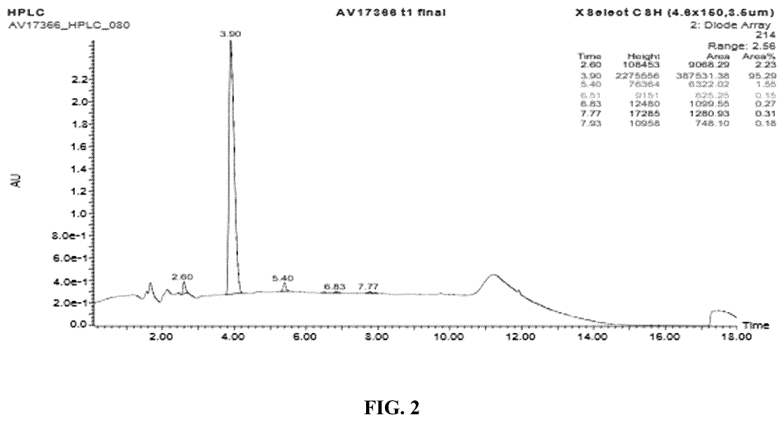

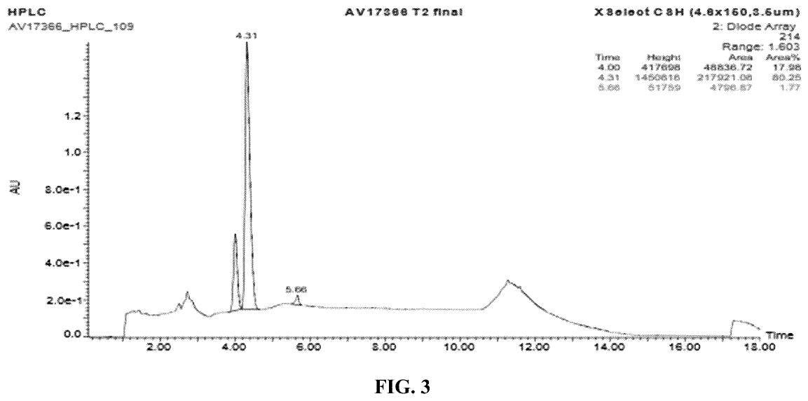

FIG. 2 shows an analytical HPLC chromatogram of residue from preparative fraction 1.

FIG. 3 shows an analytical HPLC chromatogram of residue from preparative fraction 2.

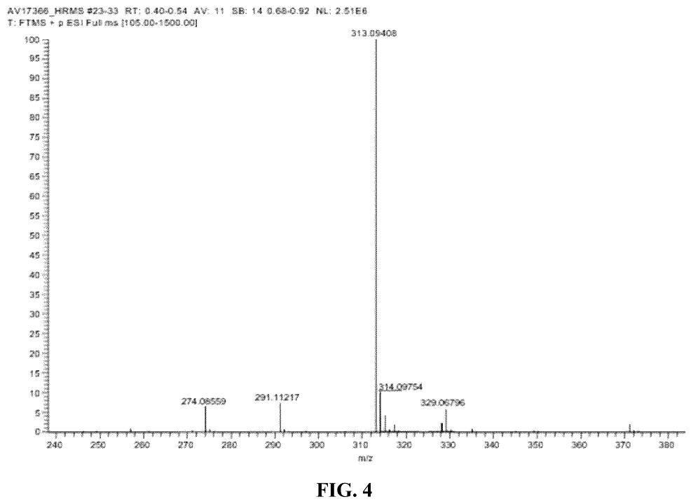

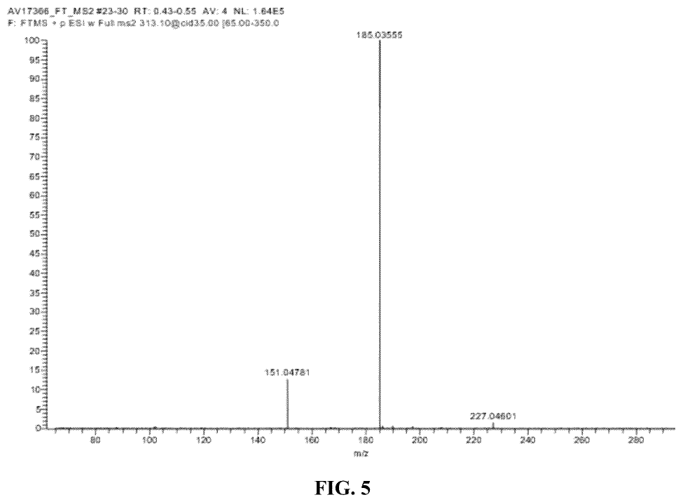

FIG. 4 shows full scan ESI+ spectrum of residue from Impurity B1.

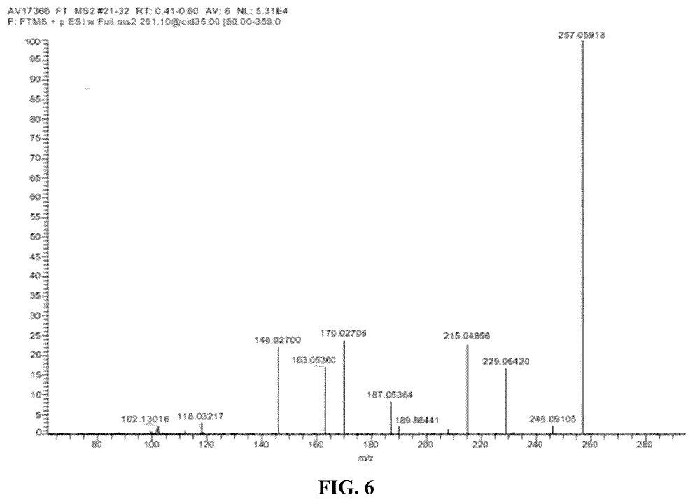

FIG. 5 shows ESI+ MS/MS of 313.1 spectrum of Impurity B1.

FIG. 6 shows ESI+ MS/MS of 291.1 spectrum of Impurity B1.

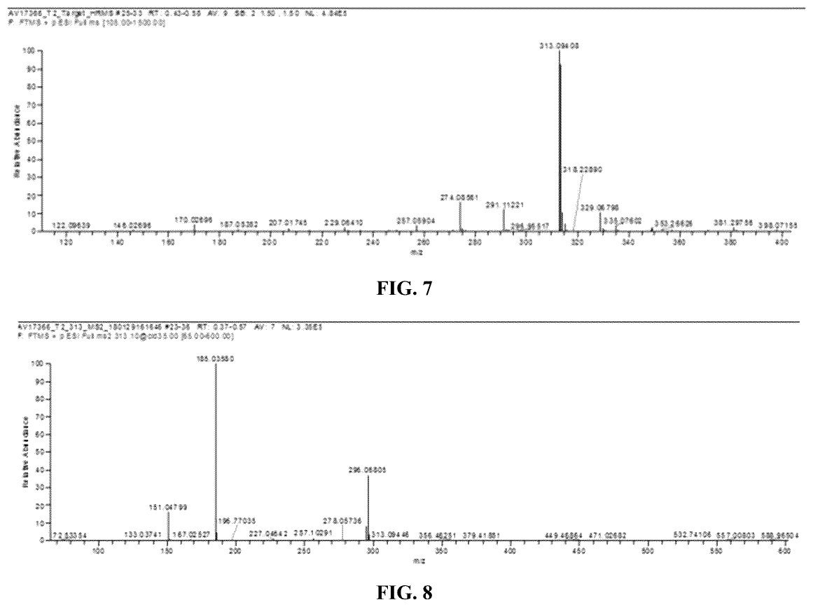

FIG. 7 shows Full scan ESI+ spectrum of enriched Impurity B2.

FIG. 8 shows ESI+ MS/MS of 313.1 spectrum of Impurity B2.

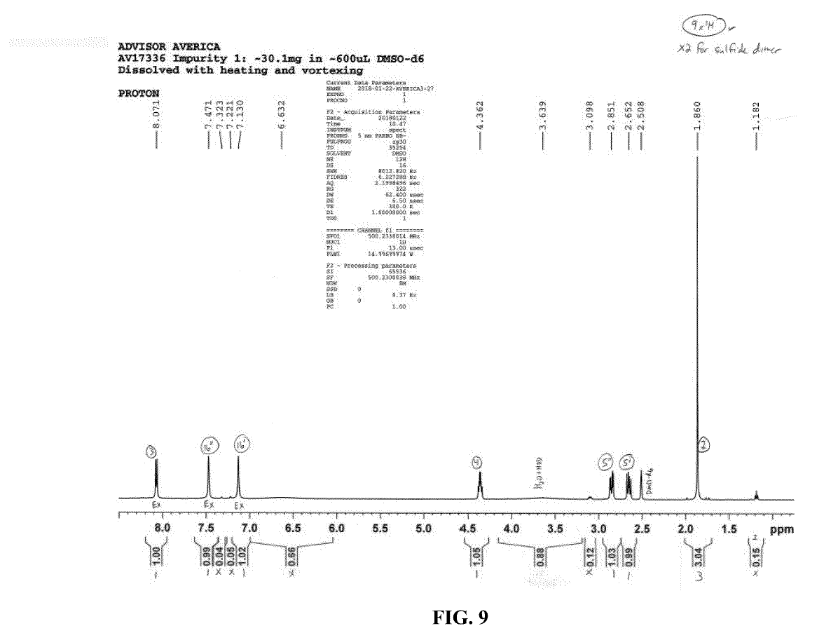

FIG. 9 shows ¹H-NMR spectrum of Impurity B1.

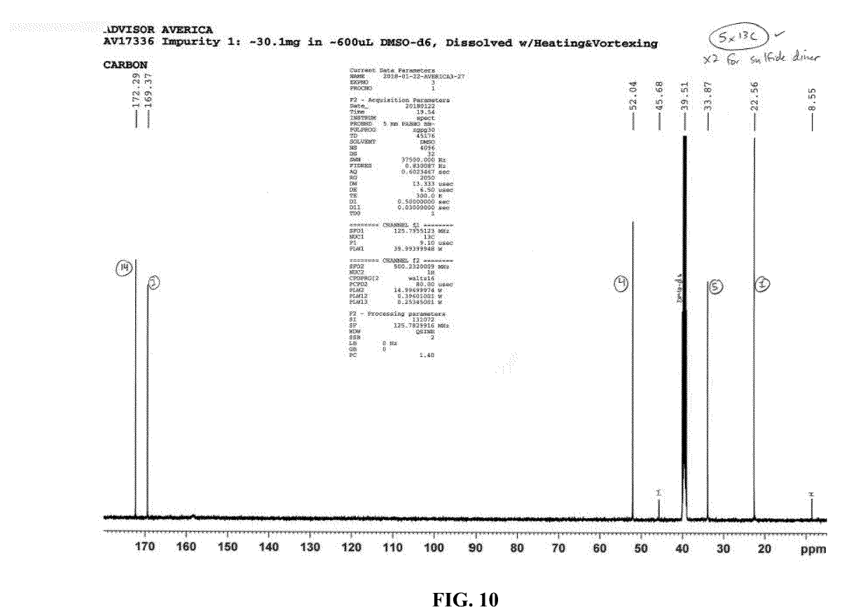

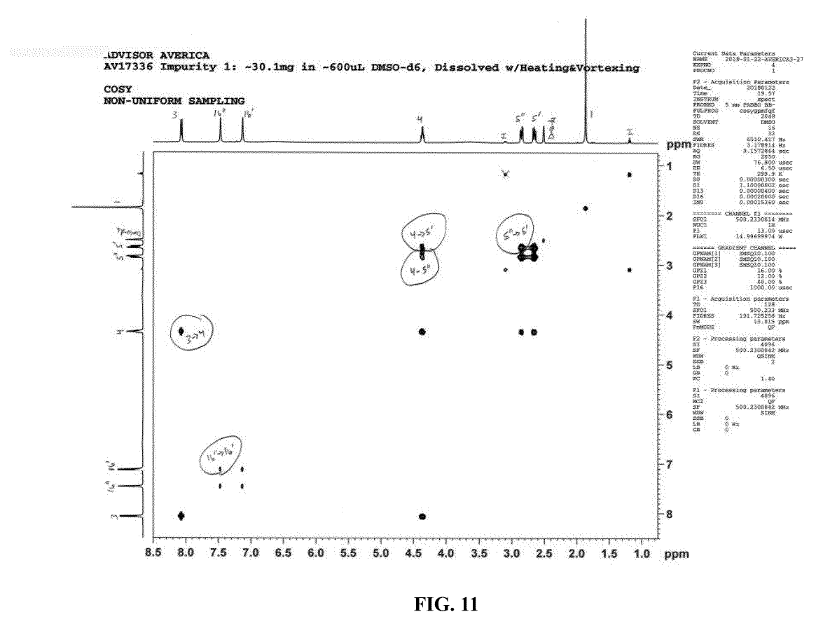

FIG. 10 shows ¹³C-NMR spectrum of Impurity B1.

FIG. 11 shows COSY NMR spectrum of Impurity B1.

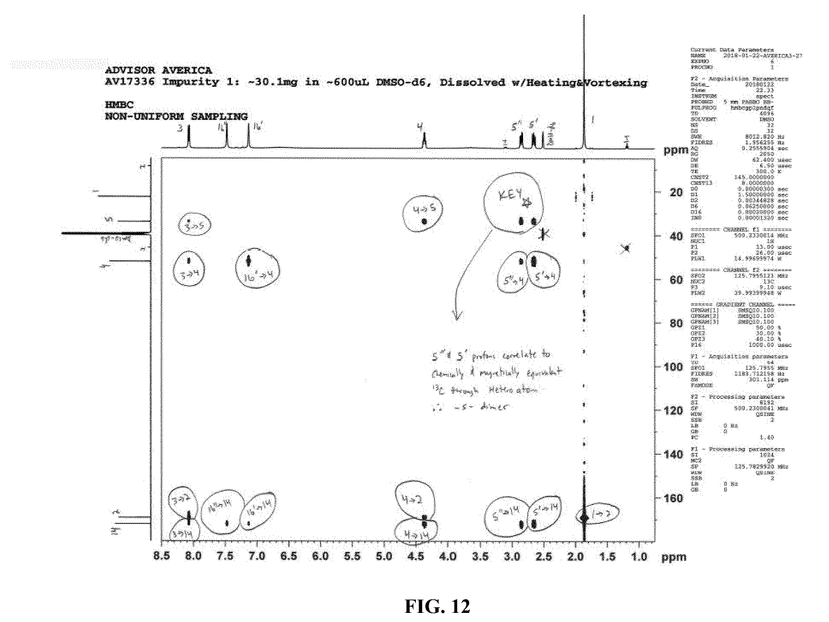

FIG. 12 shows HMBC NMR spectrum of Impurity B1.

FIG. 13 shows HSQC NMR spectrum of Impurity B1.

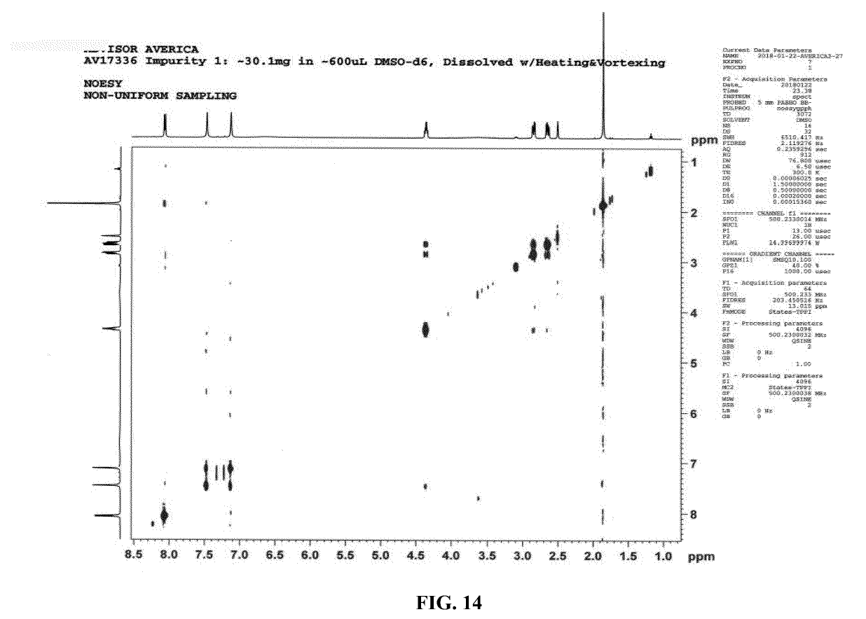

FIG. 14 shows NOESY NMR spectrum of Impurity B1.

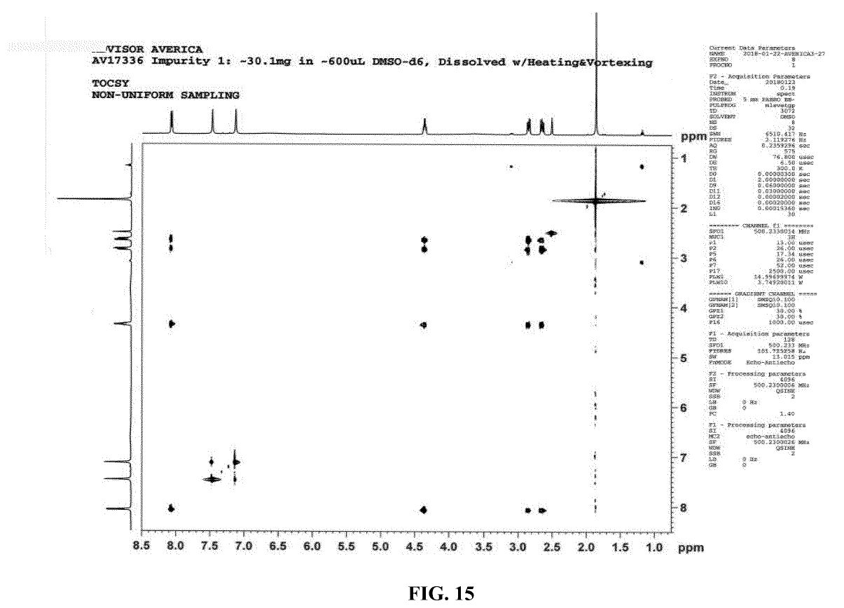

FIG. 15 shows TOCSY NMR spectrum of Impurity B1.

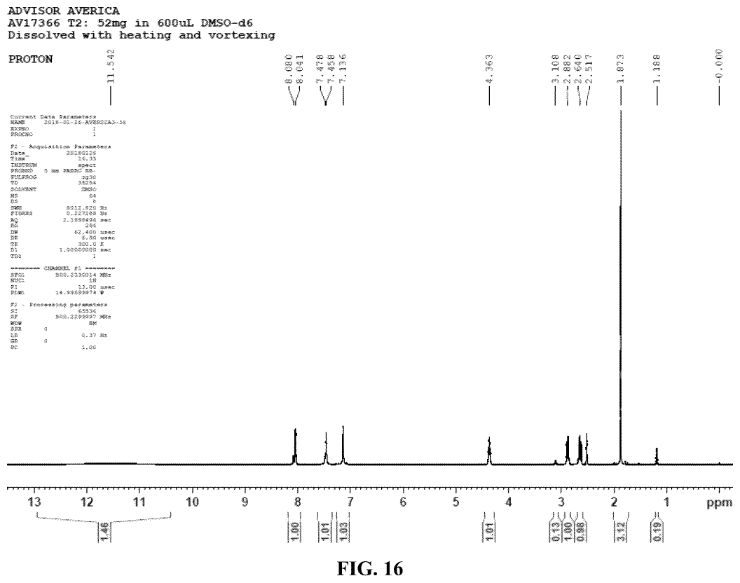

FIG. 16 shows ¹H-NMR spectrum of Impurity B2.

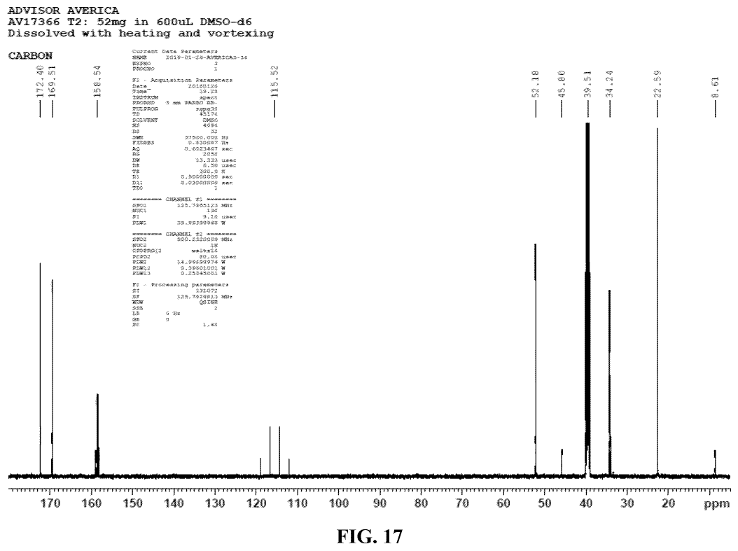

FIG. 17 shows ¹³C-NMR spectrum of Impurity B2.

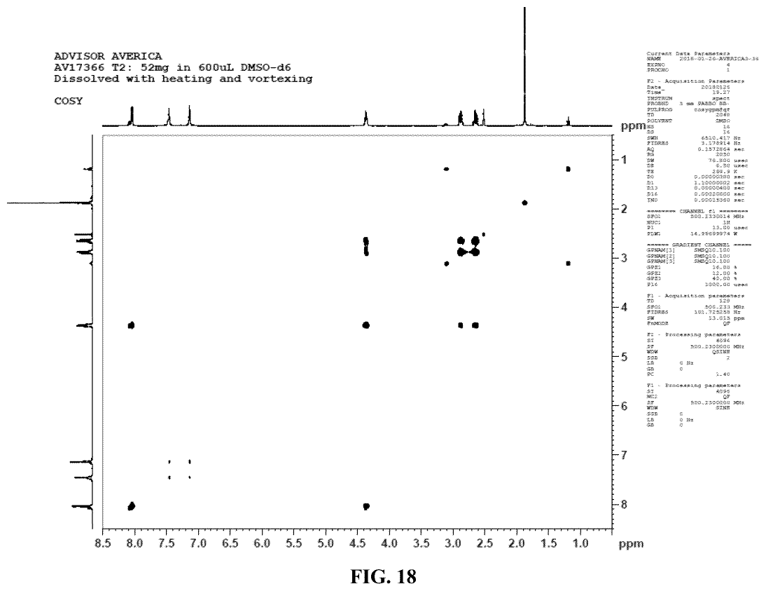

FIG. 18 shows COSY NMR spectrum of Impurity B2.

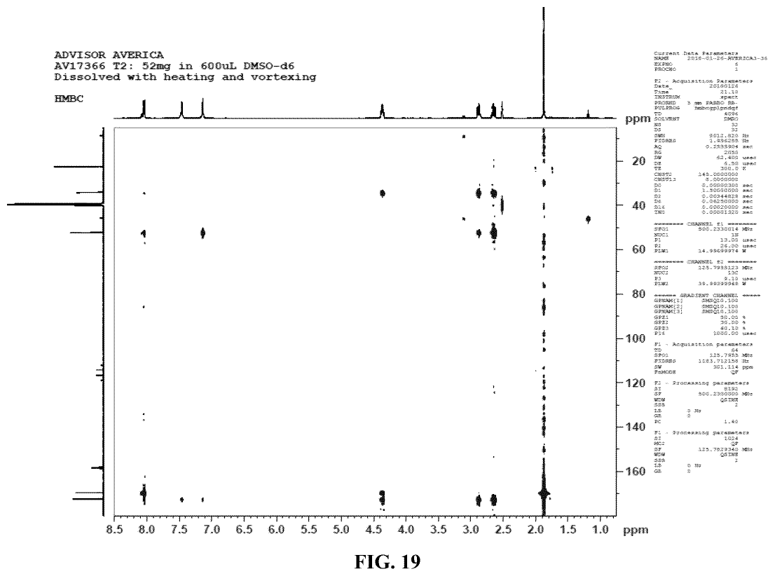

FIG. 19 shows HMBC NMR spectrum of Impurity B2.

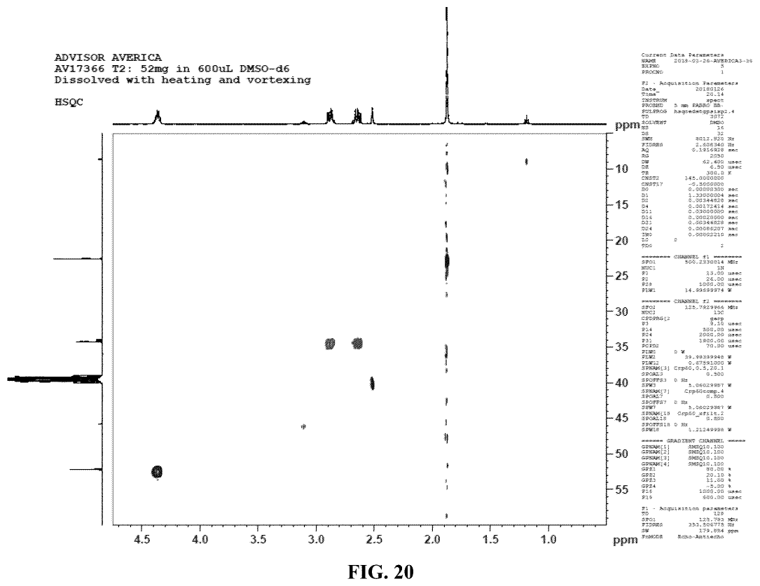

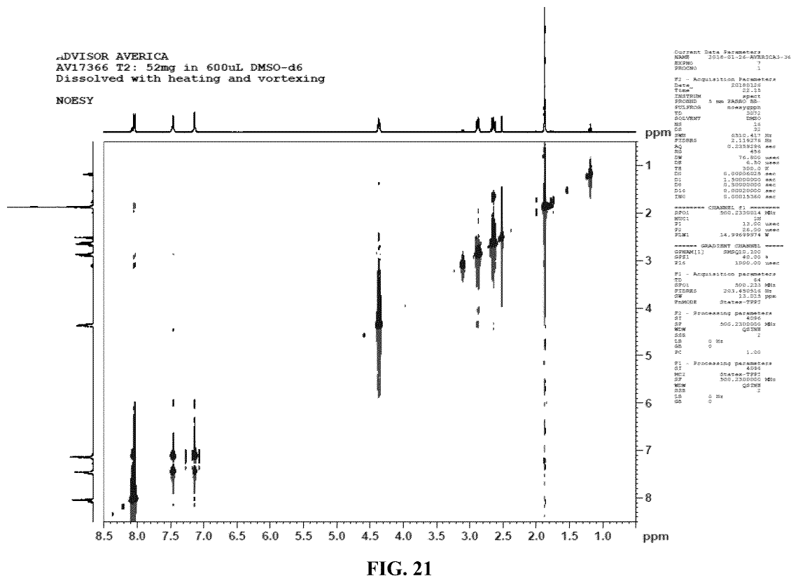

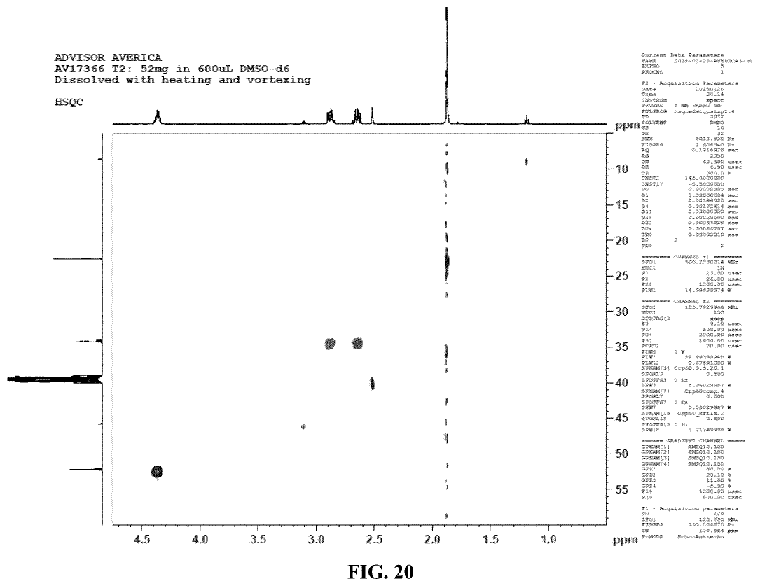

FIG. 20 shows HSQC NMR spectrum of Impurity B2.

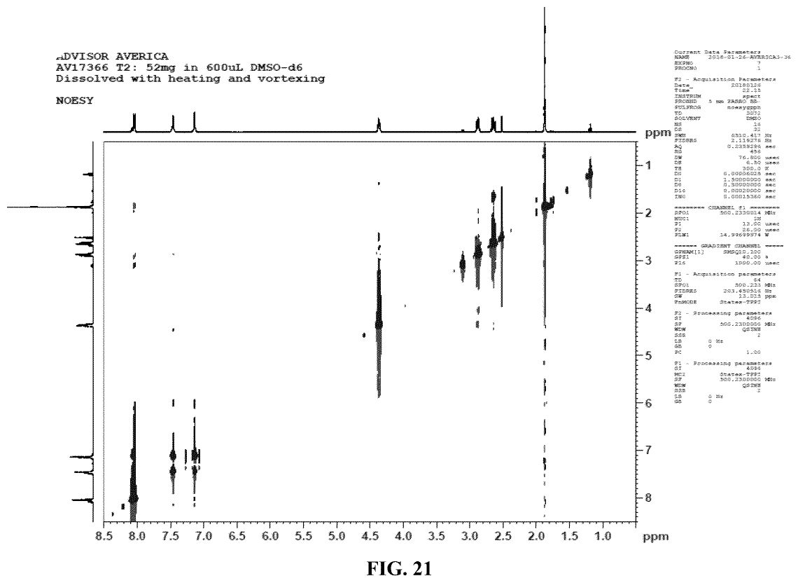

FIG. 21 shows NOESY NMR spectrum of Impurity B2.

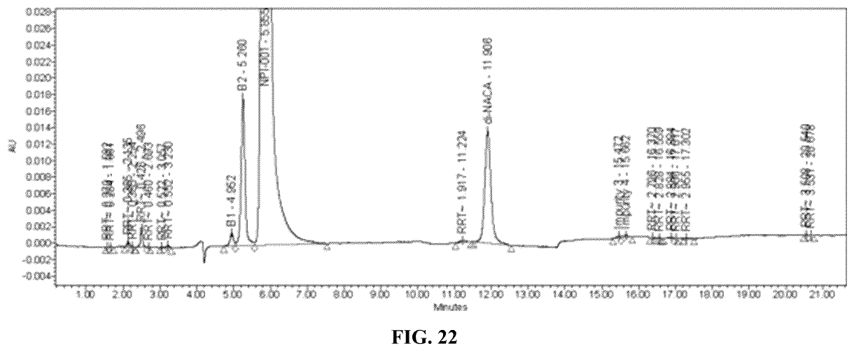

FIG. 22 shows HPLC chromatogram of impurities B1 and B2 in NPI-001 Tablets.

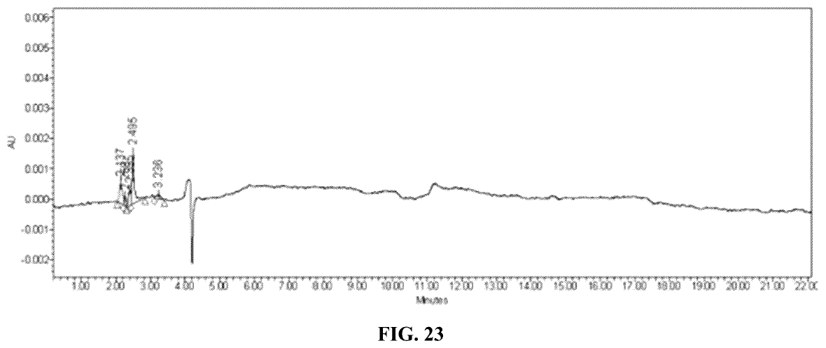

FIG. 23 shows HPLC chromatogram of Placebo showing neither Impurity B1 nor B2, and no NPI-001.

Manufacturing process of U.S. Pat. No. 10,590,073 yields N-Acetylcysteine amide (NACA, NPI-001) with greater purity, i.e., NACA (e.g., total unspecified impurities less than about 0.24%, Table 23) compared to NACA synthesized by other reported process, e.g., the process described in U.S. Pat. No. 9,763,902B2 (e.g., total unspecified impurities 1.55%, Table 24). Hence, N-Acetylcysteine amide (NACA, NPI-001) manufactured according to the process of U.S. Pat. No. 10,590,073 is considered purified and isolated N-Acetylcysteine amide, NACA or NPI-001, that is, NACA is greater than 95, 96, 97, 98, 99, 99.1, 99.1, 99.3, 99.4, 99.5, 99.6, 99.7, or even 99.76% pure. The unique impurity profile of NPI-001 (N-acetylcysteine amide, NACA) or NPI-001 formulation, specifically the presence of innocuous impurities B1 and B2, serves as an identifier, indicator, marker, and/or anticounterfeit measure that NPI-001 (N-acetylcysteine amide, NACA) or NPI-001 formulation that was manufactured according to U.S. Pat. No. 10,590,073, i.e., that N-acetylcysteine amide is manufactured by the process of Assignee to U.S. Pat. No. 10,590,073. Impurities B1 and B2 may be identified by an HPLC method. The absence of impurities B1 and B2 in an N-acetylcysteine amide-containing product indicates the N-acetylcysteine amide was manufactured by some other synthetic process, i.e., a counterfeit method. Therefore, the presence or absence of impurities B1 and B2 in NPI-001-containing products serves as an anticounterfeit measure for N-acetylcysteine amide manufactured by the process of Assignee to U.S. Pat. No. 10,590,073. The absence of impurities B1 and B2 indicates a counterfeit scenario.

### Preparation of NACA.

The present disclosure is directed to novel methods for making N-Acetyl Cysteine Amide (NACA), or diNACA, and novel intermediates thereof. More particularly, the present disclosure takes advantage of the novel intermediates to significantly, and surprisingly, increase the efficiency of preparing diNACA in both a high chemical yield, high purity, and a high enantiomeric purity. Below is the basic chemical structure of NACA.

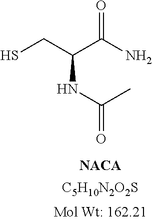

The following procedures may be employed for the preparation of the compound of the present disclosure. The starting materials and reagents used in preparing these compounds are either available from commercial suppliers such as the Aldrich Chemical Company (Milwaukee, Wis.), Bachem (Torrance, Calif.), Sigma (St. Louis, Mo.), or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supplements, Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989, relevant portions of which are incorporated herein by reference.

DiNACA is manufactured as shown in FIG. 1 of U.S. Pat. No. 10,590,073. The starting material may be the naturally occurring L-cystine with the L conformation on both cysteine subunits. In the first step the two acid groups are protected by forming the di-methyl ester of L-cystine as the dihydrochloride salt. Alternatively, the synthesis may begin with commercially acquired L-cystine dimethyl ester dihydrochloride with the L conformation on both cysteine subunits. Beginning with L-cystine dimethyl ester dihydrochloride obviates the need for thionyl chloride (typically used to convert L-cystine to L-cystine dimethyl ester dihydrochloride). In the second step, the two nitrogens on the L-cystine are reacted with acetic anhydride to give diNACME which is in essence L-cystine with both acids protected as methyl esters and both primary amine groups protected with acetyl groups. Step 3 converts the methyl ester groups to primary amides. Step 4 reductively cleaves the diNACA intermediate to give NACA.

The preparation of NACA has one starting material that contributes significantly to the backbone of the final structure: L-cystine. L-Cystine provides the chiral center and the amino acid core. Two common reagents, acetic anhydride and ammonium hydroxide, provide atoms by derivatizing the carboxylic acid and primary amine, respectively.

TABLE 1
List of Starting Materials

| Starting Material | Quality | Other |
|---|---|---|
| L-cystine | ≥98.5% pure | BSE/TSE-certification |

Control of Starting Materials. The quality of L-cystine may be controlled based on the specification provided by the manufacturer and/or incoming testing performed by the Contract Manufacturing Organization (CMO) as shown in Table 2.

TABLE 2
L-Cystine Testing

| Attribute | Test Method | Specification/Limit |
|---|---|---|
| Appearance | Visual inspection | White powder |
| ID | ¹H-NMR (USP<761>; D₂O) | Conforms to structure |
| Purity¹ | Titration | 98.5-101.0% |
| Optical Rotation | Vendor method | −215 to −225° |

¹A use test may be substituted for the purity test

Reagents and Solvents. A list of reagents and solvents used in each step of the manufacturing process is provided in Table 3 and Table 4, respectively.

TABLE 3
List of Reagents

| Step | Reagent | Quality |
|---|---|---|
| 1 | Thionyl chloride | ≥97% |
| 2, 4 | Triethylamine | ≥99% |
| 2 | Acetic anhydride | ≥99% |
| 2 | Sodium bicarbonate | ≥99% |
| 2 | Sodium sulfate (anhydrous) | — |
| 3 | Ammonium Hydroxide | 28-32% |
| 4 | Dithiothreitol | ≥98% |

TABLE 4
List of Solvents

| Step | Solvent | Quality |
|---|---|---|
| 1 | Methanol | ≥99% |
| 1, 4 | methyl t-Butyl Ether (MTBE) | ≥99% |
| 2 | Acetonitrile | ≥99% |
| 2 | Ethyl acetate | ≥99% |
| 3, 4 | EtOH | 89.0-92.0% |

In the first step, the dimethyl ester of L-cystine was formed. L-cystine and methanol were cooled to −10° C. before slowly adding thionyl chloride. The skilled artisan will recognize methanol can be substituted with other similar reactive alcohols, and/or other alternative carboxylic acid activating agents (e.g., oxalyl chloride, CDI, etc.). The slurry was then heated to reflux to produce a solution. Initially, the reaction was held for 4 hours before proceeding to the workup. Using HPLC it was found to not achieve full conversion. Holding the reaction at reflux for 16 hours proved to be effective for conversion. Upon verifying reaction completion, the solution was solvent-exchanged into ethyl ether. Depending on the method of making, other such solvent may also be used, e.g., methyl tert-butyl ether (MTBE). MTBE has proven to be an effective replacement for ethyl ether, as such the solution can be solvent-exchanged successfully into MTBE, and can also be used for washes. The white solids were filtered and washed with MTBE before drying at 45° C. under vacuum to a yield of 95-99% with a purity of 96-99%.

In Step 2, the L-cystine dimethylester dihydrochloride is converted to di-N-acetylcystine dimethylester, DiNACME. L-cystine dimethylester dihydrochloride, as a slurry in acetonitrile, is cooled to 0±5° C. To the cooled solution is first added at least 4 equivalents of triethylamine (Et₃N) followed by at least 2 equivalents of acetic anhydride while maintaining an internal temperature of ≤5° C. throughout the additions. After aging for not less than 30 minutes, reaction completion is confirmed by HPLC. To the reaction mixture is added ethyl acetate and an aqueous workup is performed. Upon completion of the aqueous workup, the organic solution is fully exchanged into ethyl acetate by vacuum distillation of the acetonitrile and replacement with ethyl acetate, several times. The product, di-N-acetylcystine dimethylester, is isolated by filtration and dried to a 73-75% yield with a purity of 93-97%. Step 2 can be accomplished in a couple of different ways. While not as efficient, the L-cystine dimethylester dihydrochloride was free-based using 300-weight percent amberlyst-A21 in 10 volumes of acetonitrile. The solution of free-based material in acetonitrile was then acetylated using 2.1 equivalents acetic anhydride and 2.5 equivalents Et₃N at room temperature for 1 hour. The crude material was concentrated and dissolved into ethyl acetate before being washed with saturated sodium bicarbonate and brine solutions. The washed organics were concentrated to dryness for a 73-75% yield with a purity of 93-97%.

Alternatively, and more efficiently, the Step 2, Et₃N after the free-basing was replaced with amberlyst-A21 for a total of 600-weight percent. Acetonitrile was increased to 15 volumes to facilitate agitation. After free-basing is complete, the acetic anhydride can be added at room temperature or below. Some impurities may be formed when reaction was performed at room temperature, as such, the reaction can be cooled to 0° C. to successfully reduce impurity formation. After reaction completion, the solution was previously concentrated to dryness. Typically, filterable solids may be preferred in certain isolation steps. As such, the acetonitrile solution was solvent-exchanged into ethyl acetate to yield filterable solids. These solids were filtered and washed with ethyl acetate before drying at 45° C. under vacuum for a 45% yield with a purity of 98%. The solvent-exchanged material can also be chilled to 0° C. for 1 hour to increase yield to 65% while achieving similar purity.

Typically, Et₃N is cheaper than amberlyst-A21, hence, the reaction can also be accomplished using only Et₃N. This was achieved by cooling the mixture of L-cystine dimethylester dihydrochloride in 15 volumes acetonitrile to 0° C. before adding 4.2 equivalents of Et₃N followed by 2.1 equivalents of acetic anhydride. Previously, using amberlyst-A21 to free-base, the hydrochloride salt formed was bound to amberlyst-A21 and filtered off of the material to yield a clear colorless L-cystine dimethylester solution. Using Et₃N as free base led to the formation of Et₃N chloride salts. These were filtered off before salting out Et₃N using 0.5 N HCl in 13% NaCl solution. At this point, the reaction was solvent-exchanged into ethyl acetate and filtered similar to previous methods. The alternative method resulted in 88-92% yield with 93-95% purity.

Using only Et₃N for Step 2 led to some loss of product before acid treatment, ineffective removal of Et₃N using acid, and degradation of material. When the Et₃N acetylation was first performed, solids were filtered before treating the solution. However, HPLC of the solids revealed the presence of desired product, Di-NACMe. Upon reaction completion, the slurry was then dissolved in 15 volumes ethyl acetate before treatment to maximize Di-NACMe in solution. Initially, 0.5 N HCl in 13% brine solution was used to treat the reaction solution. ¹H-NMR results of acid-treated product revealed significant amounts of Et₃N were still present in Step 2 product. In addition to the ineffective removal of Et₃N, the presence of acid in the material before drying degraded the material into a brown taffy when heat was applied. Since acid not only failed to remove Et₃N but also degraded material, acid was avoided. Saturated sodium bicarbonate replaced HCl to free-base Et₃N, acetic acid and HCl after reaction completion. After treating reaction solution with base to neutralize HCl, the belief was that Et₃N could be azeotroped with acetonitrile (ACN) while acetic acid stayed behind in ethyl acetate after the solvent-exchange.

The amidation of Di-NACMe into diNACA was performed in ammonium hydroxide. Initially, the solid was charged with at least 3 equivalents of 28-30% aqueous ammonium hydroxide. The solids dissolved in solution as diNACA precipitated. The slurry was agitated for 2 hours after solid formation before filtering and washing with minimal water. The solids were dried at 45° C. under vacuum. This method resulted in 60-70% yield with 70% purity. A solvent-exchange into EtOH after reaction completion further increased yield without sacrificing purity. Higher purity of the diNACA can be used to achieve a higher yield in the final reduction step. Additionally, diNACA may be recrystallized from water to further enhance the purity.

The reduction of diNACA to NACA can be accomplished using tris (2-carboxyethyl)phosphine (TCEP), thioglycolic acid, and/or dithiothreitol (DTT). One reduction method uses 1.1 eq TCEP in 15 volumes 10:1 tetrahydrofuran (THF):water, heated to reflux. Reduction by thioglycolic acid was performed using 2.5 eq thioglycolic acid. The reaction was attempted using THF and EtOH as solvents. THF reaction yielded ˜42% with a purity of 4%. EtOH reaction had a yield of 2% with purity of 30%. With the goal of using EtOH, amberlyst-A21 and Et₃N were evaluated as bases to aid reduction. Amberlyst-A21 was determined to be a better base with yields of 45% and purity of 85%. Amberlyst-A21 also reduced reaction time from 2 days in Et₃N-mediated reactions to 3 hours in amberlyst-A21-mediated reactions. Alternatively, DTT can also be used. Various possible solvents can be used, e.g., dichloromethane (DCM), methanol, isopropanol (iPrOH), EtOH and/or other alcoholic solvents. EtOH was chosen for its greater solubility of diNACA. The use of alternative reducing agents such as, but not limited to triphenylphosphine, TCEP, thioglycolic acid, etc., is contemplated herein as well as solvents such as, but not limited to, THF, EtOH, iPrOH, MeOH, DCM, water, etc. and bases, whether catalytic or stoichiometric, such as, but not limited to, Et₃N, NaOH, amberlyst-A21, etc. Initially, to effect the reduction of the disulfide bond, 1.5 equivalents Et₃N were added to diNACA dissolved in ethanol followed by 1.5 equivalents of DTT. The solution was heated to reflux and held for 2 hours before solvent-exchanging into methyl tert-butyl ether to precipitate filterable solids. Further optimization of the reaction led to the reduction of Et₃N to a catalytic 0.1 equivalents, DTT to 1.25 equivalents and reaction temperature to 62±3° C. with the effect of improving the impurity profile of the isolated NACA. This method yielded 85% of NACA with a purity of ≥98.0% (percent purity). The purity of NACA may be further enhanced via recrystallization from EtOH. No chromatography was needed.

These efforts to increase purity focused on the major impurities as measured by HPLC, namely, diNACA, DTT and cyclic DTT. Oxidation of NACA occurs when exposed to air. EtOH and MTBE used in the reduction and solvent-exchange were degassed in an effort to reduce diNACA. MTBE proved to be efficient in removing DTT and cyclic DTT, so an MTBE trituration was performed to improve purity.

diNACA purifications were performed with the goal of taking purer material through reduction for purer NACA.

Step 1: Formation of L-Cystine Dimethylester Dihydrochloride.

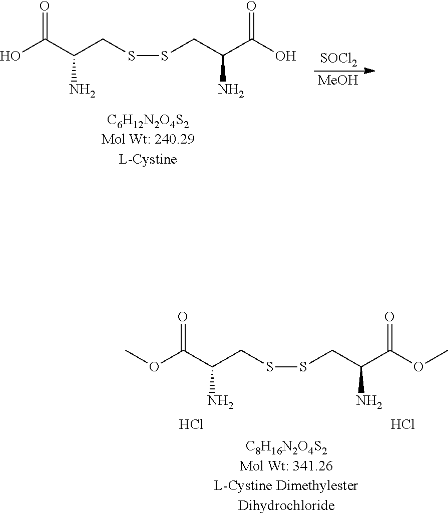

| | | | | | Weight/ |
|---|---|---|---|---|---|
| Reagents/Materials | MW | Density | Eqs. | Moles | Volume |
| L-Cystine, ≥98.5% | 240.29 | — | 1.0 | 208 | 50 kg |
| Thionyl Chloride, ≥97% | 118.97 | 1.64 | 2.41 | 504 | 60 kg |
| Methanol (MeOH), ≥99% | 32.04 | 0.79 | 12.5 vol | — | 625 L |
| Methyl-tert Butyl Ether | 88.15 | 0.74 | 8 vol | — | 400 L |
| (MTBE), ≥99% | | | | | |

Set-up: A 2000 L glass-lined reactor;

50 kg L-Cystine; and

625 L Methanol was charged to the reactor and agitated while cooling to −10° C.

60 kg Thionyl Chloride was slowly added at ≤−5° C. After addition completion, the reaction material was heated to reflux and held for 16 hours. After the reaction was verified as complete by HPLC (≤0.5% starting material), the reaction was cooled to room temperature. The reaction mixture was concentrated to 6 volumes before solvent-exchanging into 3 x8 volumes MTBE. The resulting slurry was agitated at room temperature for 1 hour before being filtered and washed with MTBE. The solids were dried at 45° C. under vacuum.

Yield: 68.15 kg (95.9%), Purity: 95.8%

Step 2: Formation of di-N-acetyl-1-cystine dimethylester (Di-NACMe)

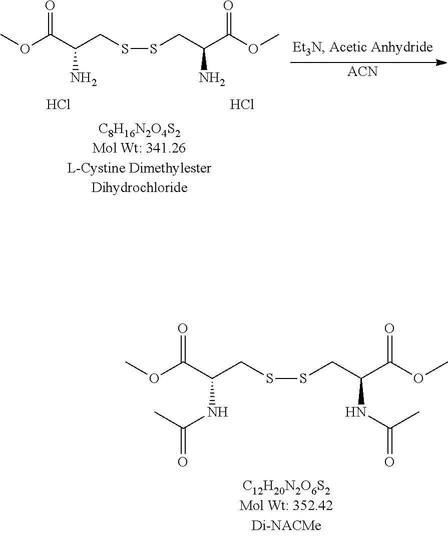

| | | | | | Weight/ |
|---|---|---|---|---|---|
| Reagents/Materials | MW | Density | Eqs. | Moles | Volume |
| L-Cystine Dimethylester Dihydrochloride, ≥95% | 341.26 | -- | 1.0 | 76.2 | 26 |
| Acetonitrile, ≥99% | 41.05 | 0.79 | 23vol | -- | 472 |
| Et3N, ≥99% | 101.19 | 0.73 | 4.2 | 316.2 | 32 |
| Acetic Anhydride, ≥99% | 102.09 | | 2.1 | 156.7 | 16 |
| Ethyl Acetate, ≥99% | 88.1 | | 41 vol | -- | 958 |

Set-up: A 800 L glass lined reactor.

26 kg L-Cystine Dimethylester Dihydrochloride and

390 L ACN is charged to the reactor and agitated while cooling to 0° C.

32 kg Et3N is added to the reactor at ≤5° C.

16 kg Acetic Anhydride is slowly added to the reactor at ≤5° C. Upon addition completion the reaction is held for 30 minutes at 5±5° C. After reaction was verified as complete by HPLC (≤0.5% starting material), 10 volumes ethyl acetate was charged to the reactor and agitated to ambient. The resulting reaction mixture was washed with 2 x2 volumes saturated bicarbonate. The aqueous layer was back extracted with 5 volumes ethyl acetate. All organics were combined and dried over sodium sulfate and polish filtered. The filtrate was concentrated to 5 volumes before azeodrying with 2 x4 volumes acetonitrile followed by a solvent-exchange into 4 x6 volumes ethyl acetate. The resulting slurry was agitated at 0° C. for 1 hour before being filtered and washed with 2 volumes ethyl acetate. The solids were dried at 25° C. under vacuum.

Average Yield: 29.56 kg (100%), Average Purity: 92.2%.

### Step 3: Formation of DiNACA.

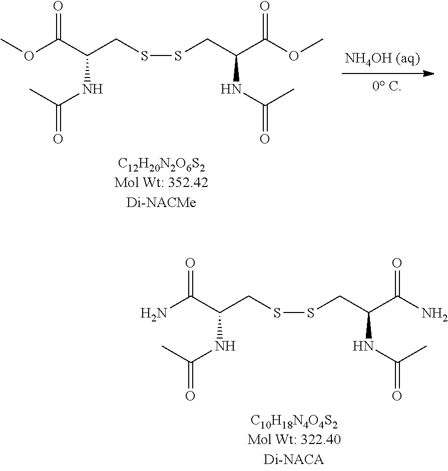

| | | | | | Weight/ |
|---|---|---|---|---|---|
| Reagents/Materials | MW | Density | Eqs. | Moles | Volume |
| Di-NACMe | 352.42 | — | 1.0 | 247 | 87.05 kg |
| 28-30% Ammonium Hydroxide | 35.05 | 0.9 | 8.44 | 2088 | 244 kg |
| Ethanol, absolute 200 proof | 46.07 | 0.79 | 17 vol | — | 1483 L |

Set-up: A 800 L glass lined reactor.

244 kg Degassed 28-30% NH4OH (aq) was cooled to 0° C. before.

87.05 kg Di-NACMe was charged to the reactor and agitated for 4 hours. After reaction was verified as complete by HPLC (≤0.5% starting material), the reaction mixture was solvent-exchanged into 3 x5 volumes degassed ethanol. The resulting slurry was agitated at 0° C. for 30 minutes before being filtered and washed with cold degassed ethanol. The solids were dried at 45° C. under vacuum.

Average Yield: 52 kg (67.1%), Average Purity: 72.0%.

### Step 3A: Purification of DiNACA.

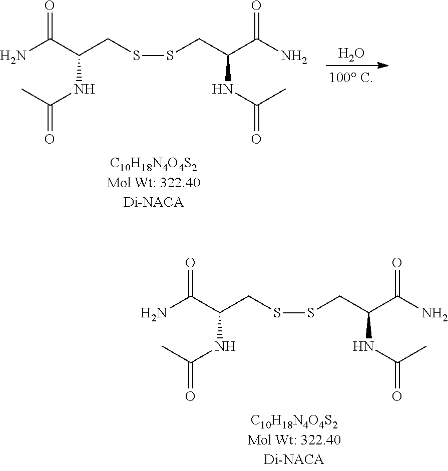 [SMILES: CC(=O)N[C@@H](CSSC[C@H](NC(C)=O)C(N)=O)C(N)=O ; O ; CC(=O)N[C@@H](CSSC[C@H](NC(C)=O)C(N)=O)C(N)=O]

| | | | | | Weight/\ |
|---|---|---|---|---|---|
| Reagents/Materials | MW | Density | Eqs. | Moles | Volume |
| DiNACA | 322.40 | — | 1.0 | 161 | 52 kg |
| Process Water, Filtered | 18.02 | 1.0 | 8 vol | — | 416 L |

Set-up: A 800 L glass lined reactor.

52 kg DiNACA and

416 L Degassed Water were charged to the flask and agitated while heating to reflux.

Upon dissolution, the reaction solution was allowed to cool overnight. The solids were filtered and washed with 2 volumes cold degassed water. The solids were dried at 45° C. under vacuum. This material may be used to continue the synthesis of NACA.

Recovery: 36 g (69%), Purity: 86.4%.

Alternate Purification of diNACA as a Final Product:

DiNACA is suspended in degassed water and heated to reflux. After cooling to ambient temperature, the product is filtered, washed with ethanol and dried to yield diNACA as a final product.

### Step 4: Formation of NACA.

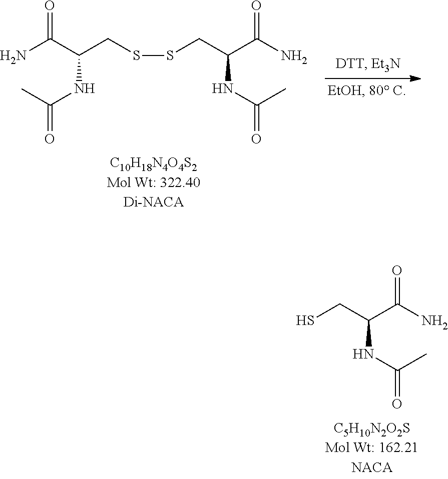

| | | | | | Weight/ |
|---|---|---|---|---|---|
| Reagents/Materials | MW | Density | Eqs. | Moles | Volume |
| DiNACA | 322.40 | — | 1.0 | 112 | 36 kg |
| Ethanol, absolute, 200 proof | 46.07 | 0.79 | 20 vol | — | 720 L |
| Et3N (TEA), ≥99% | 101.19 | 0.73 | 0.1 | 8.8 | 1.1 kg |
| Dithiothreitol, ≥98% | 154.25 | — | 1.25 | 143 | 22 kg |
| (aka 1,4-Dithiothreitol and DL- | | | | | |
| Dithiothreitol) | | | | | |

Set-up: A 800 L glass lined reactor. 720 L Degassed Ethanol, 1.1 kg Et3N, 22 kg Dithiothreitol and 36 kg of DiNACA were charged to the reactor before heating the reaction to 62±3° C. The reaction is held at temp for 2 hours. After reaction was verified as complete by HPLC (≤0.5% starting material on overloaded column), cool reaction to ambient. The reaction solution was polish filtered and concentrated to 10 volumes before solvent exchanging into 4 x10 volumes degassed MTBE. The resulting slurry was agitated overnight before being filtered and washed with 2 volumes degassed MTBE. The solids were dried @ 45° C. under vacuum. diNACA was recrystallized with ethanol.

Yield: 27.8 kg (77.2%), Purity: 98.5%.

Analytical procedures for NACA. The analytical methods for the testing of NACA drug substance are listed in Table 5. Most of the methods are USP compendial tests. The additional NACA drug substance methods (which are not compendial) include two HPLC methods for assay and impurities, and one chiral HPLC method for chiral purity. Each of the non-compendial methods is described in more detail in sections that follow.

TABLE 5
List of Analytical Procedures for NACA Drug Substance

| Test | Test Method | Description |
|---|---|---|
| Appearance | Visual | A sample of the solid material is |
| | | examined visually for form and |
| | | color. |
| ID-1 | IR | Method follows USP<197A> |
| ID-2 | ¹H-NMR | Method follows USP<761> |
| Potency/ | Calculated | Purity = (100 − % HPLC |
| Assigned | | impurities − % H₂O − % ROI − |
| Purity | | % Total Residual Solvents) |
| Organic | HPLC-Method I | Reverse phase gradient HPLC |
| | | method. |
| Impurities/ | HPLC Method II | Reverse phase gradient HPLC |
| | | method. |
| Related | | |
| Substances | | |
| Chirality | Optical Rotation | Method follows |
| | | USP<781> (c 1.00, MeOH) |
| Chiral Purity | Chiral HPLC | Chiral HPLC method |
| Residue on | USP<281> | Method follows USP<281> |
| Ignition | | |
| DSC | USP<891> | Method follows USP<891> |
| X-ray Powder | USP<941> | Method follows USP<941> |
| Diffraction | | |
| Heavy Metals | USP<233> | Method follows USP<233> |
| Residual Solvents | GC (USP<467>) | Method follows USP<467> |
| Water Content | Karl Fischer | Method follows USP<921> |
| | | version 1c |
| Microbial Limits | Microbial | Method follows USP<61> |
| | enumeration | |
| | Test for | Method follows USP<62> |
| | specified | |
| | organisms | |

HPLC Method for Purity and Related Substances. Analysis of the NACA drug substance for purity and related substances makes use of a reverse phase HPLC with an ultraviolet (UV) detector. The method is summarized in Table 6. This method is also used as the in-process method for each step to follow completion of reaction.

TABLE 6
Summary of NACA HPLC Method I
(Purity and Related Substances)

| Method | | | |
|---|---|---|---|
| Element | Description | | |
| Column | Phenomenex Synergi Hydro-RP, 4.6 × 250 mm, 4 μm | | |
| Detection | 214 nm | | |
| Column | 40° C. | | |
| Temperature | | | |
| Injection | 25 μL | | |
| Volume | | | |
| Flow Rate | 1.0 mL/minute | | |
| Mobile Phase A | 0.02% H₃PO₄ in H₂O | | |
| Mobile Phase B | Acetonitrile (ACN) | | |
| | Time | | |
| Gradient | (min) | % A | % B |
| | 0.0 | 100 | 0 |
| | 15.0 | 90 | 10 |
| | 25.0 | 0 | 100 |
| | 30.0 | 0 | 100 |
| | 30.1a | 100 | 0 |
| | 35.0 a | 100 | 0 |

| | |
|---|---|
| | (a) Equilibration time-no integration |
| System | 1. Specificity: No significant interference in the |
| Suitability | blank chromatogram at retention times of interest. |
| | 2. The S/N of the NACA peak in the 0.03% |
| | sensitivity solution must be ≥10 |
| | 3. The % RSD of the RT and peak area of NACA in |
| | the 5 injections of the first sample must be ≤2.0%. |
| | 4. The recovery of one standard prep (average of |
| | all injections) versus a second standard prep |
| | (average of all injection) must be 100.0 ± 2.0%. |

HPLC Method for Impurities B1 and B2. Method-I did not always detect impurities B1 and B2. A second method, Method-II, was developed to reliably quantitate these two impurities. The method is summarized in Table 7.

TABLE 7
Summary of NACA HPLC Method II (Impurities B1 and B2)

| Method Element | Description | | |
|---|---|---|---|
| Column | Agilent Zorbax SB-Aq, 4.6 × 250 mm, 5 μm | | |
| Detection | 214 nm | | |
| Column Temperature | 40° C. | | |
| Injection Volume | 25 μL | | |
| Flow Rate | 1.0 mL/minute | | |
| Mobile Phase A | 0.02% H₃PO₄ in H₂O | | |
| Mobile Phase B | Acetonitrile (ACN) | | |
| | Time | | |
| Gradient | (min) | % A | % B |
| | 0.0 | 100 | 0 |
| | 5.0 | 100 | 0 |
| | 20.0 | 0 | 100 |
| | 25.0 | 0 | 100 |
| | 25.01 | 100 | 0 |
| | 35.0 | 100 | 0 |

| | |
|---|---|
| System | 1. Specificity: No significant interferences in the |
| Suitability | blank chromatogram at retention times of interest. |
| | 2. The S/N of the NACA peak in the 0.03% |
| | sensitivity solution must be ≥10 |
| | 3. The % RSD of the RT and peak area of NACA |
| | in the 5 injections of the first sample must |
| | be ≤2.0%. |

Chiral HPLC Method for Measuring Chiral Purity of NACA. A chiral method was developed to assess the chiral purity of NACA. The method is summarized in Table 8.

TABLE 8
Summary of Chiral HPLC Method for NACA

| Method Element | Description |
|---|---|
| Column | Diacel Chiralpak IC-3, 4.6 × 150 mm |
| Detection | 217 nm |
| Column Temperature | 35° C. |
| Injection Volume | 20 μL |
| Flow Rate | 0.8 mL/minute |
| Mobile Phase (isocratic) | 0.05% H₃PO₄ in 1:1 n-hexane:IPA |
| System | 1. Specificity: No significant interferences |
| Suitability | in the blank chromatogram at retention times |
| | of interest. |
| | 2. The resolution between the enantiomers is |
| | sufficient to allow for accurate integration of |
| | both peaks. |
| | 3. The S/N of the NACA peak in the 0.03% |
| | sensitivity solution must be ≥10 |
| | 4. The % RSD of the RT and peak area of |
| | NACA in the 6 injections of the first sample |
| | must be ≤2.0%. |

Preparation of D-NACA. D-Cystine was obtained from a commercial vendor. D-Cystine was dissolved in water and pH was adjusted to pH 9-10 with NaOH. Acetic anhydride was added dropwise at 0° C. and pH maintained at 9-10. Solution was stirred for 4 hours after which it was acidified to pH ˜2 with HCl. The solvent was evaporated under reduced pressure. 20 mL MeOH was added to the flask to dissolve contents. Solution was filtered. Filtrate was concentrated and evaporated. Thin layer chromatography (MeOH:DCM:HOAc, 1:8:1) indicated loss of starting material and appearance of a single new peak for D-acetylcystine. D-Acetylcystine was charged to a round-bottomed flask with 50 mL MeOH to which was added 0.35 mL concentrated H₂SO₄ via syringe, dropwise, at ambient temperature, with agitation. Solution turned turbid. IPC by TLC (MeOH:DCM, 1:9] showed no starting material. Solvent was evaporated. Fraction was neutralized with NaHCO₃ (saturated), extracted with DCM (50 ml twice), washed with water, dried over Na₂SO₄, filtered, purged with nitrogen and concentrated under reduced pressure to yield a net weight of 7.8 g white solid, formed in the refrigerator. N-acetylcysteine methyl ester was charged into a 3-necked, round-bottomed flask with nitrogen bubbler, and magnetic stirrer. Ammonium hydroxide at ambient temperature was added and purged with nitrogen through reaction mixture and agitated at ambient temperature. Solvent was evaporated under vacuum and a white solid formed. Ethanol was added and heated to form clear solution and left to stand overnight. White solid crystallized, was filtered, washed with ethanol and purified by column chromatography.

### Oral Solution of NACA.

A study was conducted to determine the solubilization of NACA in ORA-SWEET® (Ora-Sweet is a commercially available syrup vehicle containing water, sucrose, glycerin, sorbitol, flavoring, buffering agents (citric acid and/or sodium phosphate), methyl paraben and potassium sorbate, pH 4.2 manufactured by Paddock Laboratories, Inc., Minneapolis, Minnesota). HPLC equipment and glass containers and stirrers were utilized.

NACA at 80 mg/ml did not readily dissolve in ORA-SWEET, rather it required stirring for 20-30 minutes to achieve a solution. However, NACA was readily dissolved in water with shaking for 30 seconds, followed by dilution with an equal volume of ORA-SWEET with shaking for 20-30 seconds. Therefore, NACA was dissolved in 50 mL water followed by 50 mL Ora-Sweet, shaken to dissolve in an opaque plastic bottle with closure and provided to subject for self-administration (oral ingestion).

NACA oral solution was also be prepared as 100 ml oral solution in ORA-SWEET. Doses can be achieved ranged from 250 mg to 4000 mg/day/patient. The NACA dissolution in water followed by dilution with ORA-SWEET was found optimal for compounding. ORA-SWEET is a pale pink solution with a cherry syrup flavor. NACA has a mild sulfur odor and a bitter taste (like burned sesame seeds). When dissolved in ORA-SWEET, the odor and taste were masked.

The following instructions for preparation of NACA Oral Solution were developed:

Weigh NACA [either 250, 750, 1500, 3000 or 4000 mg (±1 mg), as appropriate for the particular dose group] and place into a 125 mL (approximately) capacity opaque high density polyethylene, labeled (see Table 9) bottle with opaque polypropylene screw cap.

TABLE 9
Container/closure for NACA Oral Solution Used for Phase 1 Study

| Component | Description |
|---|---|
| Bottle | 125 mL opaque white high density polyethylene bottle |
| Cap | Polypropylene opaque white cap |
| Label | Pharmacy approved label |

Measure 50 mL of Purified Water and pour into each bottle containing NACA and shake vigorously by hand (at least 30 seconds) to dissolve.

A subject drinks entire solution, followed by two 20-ml rinses of the container with water, which are also drank.

Tablet or Capsule of NACA for clinical phase I or clinical phase II trials:

The qualitative composition for NACA Tablets is presented in Table 10.

TABLE 10
Qualitative Composition of NACA Tablets

| Component | Quality Standard |
|---|---|
| NACA | Nacuity |
| Lactose | NF |
| Microcrystalline Cellulose | NF |
| Croscarmellose Sodium | NF |
| Stearic acid | NF |

NACA Tablets, 250 mg, are formulated as an immediate release drug product. A roller-compacted blend containing 250 mg NACA is compressed into round, biconvex tablets.

The formulation use to manufacture NACA Tablets is a roller compacted blend of common excipients (Table 11). The blend is compressed into round, biconvex shaped tablets.

TABLE 11
Quantitative Composition of NACA Tablets

| | mg/tablet | | | Quality |
|---|---|---|---|---|
| Component | weight | % | Function | Standard |
| NACA | 250.0 | 50 | Drug | Nacuity |
| | | | substance | |
| Lactose | 42.875 | 8.575 | Filler | NF |
| Microcrystalline | 177.125 | 35.425 | Filler | NF |
| Cellulose | | | | |
| Croscarmellose | 25 | 5.0 | Disintegrant | NF |
| Sodium | | | | |
| Stearic acid | 5 | 1.0 | Lubricant | NF |
| TABLET WT | 500 | 100 | — | — |

### Type of Container and Closure for Dosage Form.

NACA Tablets, 250 mg, are packaged in high density polyethylene (HDPE) bottles with foil induction seal and a white polyproplylene screw-top closure. Excipients used for the formulation meet compendial standards. Lactose functions as a filler. Microcrystalline cellulose functions as a filler. Croscarmellose Sodium functions as a disintegrant. Stearic Acid functions as a lubricant. Excipients were screened by assessing the stability of NACA in mixtures with each excipient. Mixtures of NACA with either microcrystalline cellulose, lactose monohydrate, croscarmellose sodiumandcrospovidone/Kollidon CLandhydroxypropyl cellulose exhibitedless degradation of NACA compared to other excipients (Table 12). Hydroxypropyl cellulose exhibited greater levels of impurities than the other lead excipients (data not shown). Based on these results as well as the collective experience of the Formulators, microcrystalline cellulose, lactose monohydrate, croscarnellose sodium and steric acid were chosen as excipients for NACA Tablets.

TABLE 12
Stability results for NACA/excipient mixtures
after 4 weeks at 40° C./75% RH

| | | Ratio of | % |
|---|---|---|---|
| Sample ID | Excipient | API:Excipient | Assay |
| SPt: 17004Q4; P | NA | 1:0 | 95.3 |
| SPL-1700405-P | Microcrystalline Cellulose | 1:1 | 94.9 |
| | PH 102 | | |
| SPL-1700406-P | Lactose Monohydrate | 1:1 | 93.2 |
| | (Fastflo) | | |
| SPL-1700407-P | Croscarmellose Sodium | 4:1 | 93.6 |
| SPL-1700408-P | Crospovidone/Kollidon CL | 4:1 | 94.4 |
| SPl-1700409-P | Sodium Lauryl Sulfate | 4:1 | 26.2 |
| SPL-1700410-P | Colloidal Silicon Dioxide | 4:1 | 92.7 |
| SPL-1700411-P | Magnesium Stearate | 9:1 | 77.5 |
| SPL-1700412-P | Sodium Stearyl Fumarate | 9:1 | 86.7 |

Tables 13 and 14 were prepared. The use of roller compaction of formulations of NACA with various excipients yielded powders with acceptable flow properties. Roller compaction followed by compression yielded tablets with acceptable properties based on friability and hardness. The clinical formulation was selected based on acceptable 2-week stability data.

Dry blending of formulations of NACA with various excipients yielded poorly flowing powders. Dry blending followed by compression yielded tablets that were unsatisfactory based on friability and hardness.

The use of roller compaction of formulations of NACA with various excipients yielded powders with acceptable flow properties. Roller compaction followed by compression yielded tablets with acceptable properties based on friability and hardness.

TABLE 13
Finished Prototype NACA Tablets by Roller Compaction

| | | Packaging |
|---|---|---|
| Batch Formulation | Product Batch # | Configurations* |
| NAC1G50%0401 | NAC1T250mg0401A | 1 |
| Low Roller Compaction | NAC1T250mg0401B | 2 |
| Force (3 kN) | | |
| NAC1G50%0402 | NAC1T250mg0402A | 1 |
| High Roller Compaction | NAC1T250mg0402B | 2 |
| Force (6 kN) | | |
| NAC1G50%0501 | NAC1T250mg0501A | 1 |
| Low Roller Compaction | NAC1T250mg0501B | 2 |
| Force (3 kN) | | |
| NAC1G50%0502 | NAC1T250mg0502A | 1 |
| High Roller Compaction | NAC1T250mg0502B | 2 |
| Force (6 kN) | | |

*Packaging Configuration 1 (With Desiccant):
Bottle: 60 CC 33/400 W-HDPE ROUND BTL
Cap: 33 mm SECURX with SG-75M liner
Desiccant: Desiccant Canister Silica Gel 2GM
Fill: 20 tablets per bottle
*Packaging Configuration 2 (Without Desiccant):
Bottle: 60 CC 33/400 W-HDPE ROUND BTL
Cap: 33 mm SECURX with SG-75M liner
Fill: 20 tablets per bottle

TABLE 14
NACA Tablet Prototype Batch Formulations by Roller Compaction

| Component | | | | | | | |
|---|---|---|---|---|---|---|---|
| NAC1G50% | | NAC1G50% | | NAC1G50% | | NAC1G50% | |
| 0401 mg/ | | 0402 mg/ | | 0501 mg/ | | 0502 mg/ | |
| tablet weight | | tablet weight | | tablet weight | | tablet weight | |
| Low Force | | High Force | | Low Force | | High Force | |
| g | % | g | % | g | % | g | % |

INTRAGRANULAR

| | | | | | | | | |
|---|---|---|---|---|---|---|---|---|
| NACA | 750 | 50 | 750 | 50 | 750 | 50 | 0 | 0 |
| NACA Direct | 0 | 0 | 0 | 0 | 0 | 0 | 0 | 71.42 |
| Blend 70% | | | | | | | | |
| (NAC1B70% 01) | | | | | | | | |
| Lactose | 330 | 22 | 330 | 22 | 128.6 | 8.57 | 857.0 | 0 |
| Microcrystalline | 330 | 22 | 330 | 22 | 128.6 | 8.57 | 0 | 0 |
| Cellulose | | | | | | | | |
| Croscarmellose | 75 | 5 | 75 | 5 | 53.6 | 3.57 | 0 | 0 |
| Sodium | | | | | | | | |
| Stearic Acid | 7.5 | 0.5 | 7.5 | 0.5 | 10.6 | 0.71 | 0 | 0 |

EXTRAGRANULAR

| | | | | | | | | |
|---|---|---|---|---|---|---|---|---|
| Microcrystalline | 0 | 0 | 0 | 0 | 402.9 | 26.86 | 322.3 | 26.86 |
| Cellulose | | | | | | | | |
| Croscarmellose | 0 | 0 | 0 | 0 | 21.4 | 1.43 | 17.2 | 1.43 |
| Sodium | | | | | | | | |
| Stearic Acid | 7.5 | 0.5 | 7.5 | 0.5 | 4.3 | 0.29 | 3.5 | 0.29 |
| Total | 1500 | 100 | 1500 | 100 | 1500 | 100 | 1200 | 100 |

*NAC1B70% 01 = 70% NACA + 12% Lactose + 12MCC + 5% Croscarmellose Sodium + 1% Stearic Acid

### NACA Tablet Dry Blend Formulation Development.

NACA Tablet formulations (Table 15) were dry blended and directly compressed. The flow of formulation from the hopper to the press was not uniform. Also, the resulting tablets suffered poor friability, hardness and capping. As a result dry blending was abandoned.

TABLE 15
Composition of Prototype NACA Tablet Dry Blend Formulations.

| | Prototype Batch Composition | | | |
|---|---|---|---|---|
| | Batch NAC1B50%0101 | | Batch NAC1B50%201 | |
| Component | g | % | g | % |
| NACA | 600 | 50 | 600 | 50 |
| Lactose | 528 | 44 | — | — |
| Microcrystalline | — | — | 528 | 44 |
| Cellulose | | | | |
| Croscarmellose | 60 | 5 | 60 | 5 |
| Sodium | | | | |
| Stearic Acid | 12 | 1 | 12 | 1 |
| Total | 1200 | 100 | 1200 | 100 |

The formulations described above can also be used as a dry blend for filling into capsules.

The relation between micronization conditions and an initial increase of the degradation product diNACA was further investigated. Comparing stainless steel and zirconium oxide grinding jars gives a clear indication that steel samples undergo a time dependent increase of the degradation product diNACA. In contrast, using zirconium oxide jars and balls did not lead to an initial increase of the degradation product diNACA. Therefore, the zirconium oxide milling process was further investigated and successfully optimized regarding particle size distribution, milling parameters and impurity profile for the 1% NACA formulation. Zirconium oxide milling process was investigated and successfully optimized regarding particle size distribution, milling parameters and impurity profile for the 1% NACA formulation. The particle size distribution by laser diffraction analysis was x10=1.3 μm, x50=4.7 μm and x90=12.3 μm. Batches were prepared and found to be stable for up to 4 weeks at ambient temperature.

### Single Crystal X-Ray Diffraction.

The absolute structure of NACA has been determined by single crystal X-ray diffraction from suitable crystals grown from cooling of a saturated 2-propanol NACA solution to ambient conditions. Single crystal analysis of crystals clearly shows that the material is NACA with the expected bond connectivity. The absolute stereochemistry has been proved in the crystal with excellent confidence, as confirmed by the Flack parameter being −0.02(3). The density of the material is high, reducing the risk that a more stable polymorph is even possible and the hydrogen bonding network observed satisfies the expected functionality observed in NACA. The predicted XRPD from the SC-XRD data is consistent with the Form 1 material, indicating that the crystal was representative of the bulk material. Data was collected and found to be twinned, therefore, was refined accordingly using HKLF5 and BASF commands, locating a two-component twin with BASF scales 0.7271(10):0.2729(10) in the Monoclinic space group P21 where two complete formula units of NACA were found in the asymmetric unit only. No disorder was noted in the final structure with final a R1 [I>2σ(I)] of 3.30% obtained with Flack parameter of ˜0.02 with e.s.d 0.03 determined using 1549 quotients that is suitable to accurately determine the IUPAC name of NACA as 2R)-2-(acetylamino)-3-sulfanylpropanamide (=N-acetyl-L-cysteine amide=(R)-2-acetylamino)-3-mercapto-propamide).

NACA, Form 1 overall structure quality factor: 1.

Where:
1. Strong data set, no disorder, R1 ˜4%. Publishable quality.
2. Good data set, contains some minor disorder, R1 ˜6%. Publishable quality.
3. Average data set and/or easily modelled disorder or twinning. Publishable with care.
4. Weak data and/or major disorder or twinning that is not easily modelled. Publishable in some cases.
5. Very weak data and/or unexplained features of data or model. Not of publishable quality.

### Polymorphism.

A detailed polymorph screen of NACA (NACA) (NACA) has been performed using a variety of solvents and experimental conditions. During the primary screen, the most common solid form observed was pattern 1 (Table 16).

TABLE 16
Crystallographic parameters and refinement indicators of NACA, Form 1.
NACA, Form 1

| Empirical formula | C₅H₁₀N₂O₂S |
|---|---|
| Formula weight | 162.21 |
| Temperature/K | 120(1) |
| Crystal system | Monoclinic |
| Space group | P2₁ |
| a/Å | 7.2832(2) |
| b/Å | 7.5542(2) |
| c/Å | 13.9686(4) |
| α/° | 90 |
| β/° | 98.6983(15) |
| γ/° | 90 |
| Volume/Å³ | 759.70(4) |
| Z, Z′ | 4, 2 |
| ρ_{calc} g/cm³ | 1.418 |
| μ/mm⁻¹ | 0.369 |
| F(000) | 344.0 |
| Crystal size/mm³ | 0.384 × 0.207 × 0.131 |
| Radiation | MoKα (λ = 0.71073) |
| 2Θ range for data collection/° | 2.95 to 56.582 |
| Index ranges | −9 ≤ h ≤ 9, −10 ≤ k ≤ 10, |
| | −18 ≤ l ≤ 18 |
| Reflections collected | 5810 |
| Independent reflections | 5810 [Rᵢₙₜ = 0.0580, R_{sigma} = 0.0288] |
| Data/restraints/parameters | 5810/1/186 |
| Goodness of Fit | 1.057 |
| Final R indexes [I > 2σ (I)] | R₁ = 0.0330, wR₂ = 0.0869 |
| Final R indexes [all data] | R₁ = 0.0350, wR₂ = 0.0900 |
| Δρmax, Δρmin/e Å⁻³ | 0.47/−0.53 |
| Flack Parameter | −0.02(3) |

R₁ = (Σ |Fₒ| − |F_{c}|)/Σ |Fₒ|); wR₂ = {Σ [_{W}(Fₒ² − F_{c}²)²]/Σ [w(F_{c}²)²]}^{1/2}; S = {Σ [w(Fₒ² − F_{c}² )²]/(n − p)}^{1/2.}

Several experiments yielded diffractogram patterns that were different or, more commonly, had extra peaks observed. The extra peaks would indicate the presence of another form, albeit not in a pure phase.

Attempts to reproduce these forms failed using both crash cooling, evaporation and anti-solvent addition. Analysis of these attempts by NMR showed that the material was still predominately the NACA material and that it had not oxidized to diNACA. The lack of reproducibility of these potential forms is good evidence for their lack of stability. This study has clearly demonstrated that the NACA material exists as Form 1 and that other forms are difficult, if not impossible, to produce.

Approximately 80 mg of NACA was weighed into each of 24 vials. The solvents listed below were added to the appropriate vials. The quantities added were calculated (based on solubility studies) to dissolve approx. 60% of the material. These mixtures were temperature cycled between ambient and 40° C., in 4 hour cycles, for 72 hours. Solids isolated from the slurries are tested by XRPD. The resulting saturated solutions were separated into three separated vials for crash cooling, anti-solvent addition and evaporation experiments.

TABLE 17
List of Solvents Used in the Primary Polymorph Screen

| | Solvent |
|---|---|
| 1 | Acetone |
| 2 | Acetone/water (80:20) |
| 3 | Acetone/Heptane (75:25) |
| 4 | Acetonitrile |
| 5 | Acetonitrile/water (80:20) |
| 6 | 1-Butanol |
| 7 | 1,2-Dimethoxyethane |
| 8 | 1,4-Dioxane |
| 9 | Dioxane/water (80:20) |
| 10 | Ethanol |
| 11 | Ethanol/water (80:20) |
| 12 | Ethanol/heptane (75:25) |
| 13 | Ethyl Formate |
| 14 | Isopropyl acetate |
| 15 | Ethyl acetate |
| 16 | Methanol |
| 17 | Methanol/water (80:20) |
| 18 | Methyl Ethyl ketone |
| 19 | Nitromethane |
| 20 | 1-Propanol |
| 21 | 2-Propanol |
| 22 | 2-Propanol/water (80:20) |
| 23 | Tetrahydrofuran |
| 24 | Water |

Liquid Chromatography with Mass Spectrometric Detection
Column Temperature: 30° C.
Mobile Phase A: 0.1% v/v Formic in Water
Mobile Phase B: 0.1% v/v Formic in Acetonitrile
Diluent: Mobile Phase A: 50:50 Water: Acetonitrile
Flow Rate: 1.0 mL/min
Runtime: 25 minutes
Injection Volume: 10 μL
Detection: 190-400 nm
Gradient:

| Time (minutes) | Solvent B [%] |
|---|---|
| 0 | 0 |
| 12 | 10 |
| 15 | 100 |
| 15.1 | 100 |
| 25 | 0 |

Instrument: LCQ Advantage Ion Trap MS
Sample concentration: 1 mg/ml, +ve ion mode by infusion
Source voltage (kV): 4.50
Source current (μA): 80.00
Sheath gas flow rate: 20.00
Aux/Sweep gas flow rate: 0.00
Capillary voltage (V): 8.00
Capillary temp (° C.): 200
Tube lens (V, Sp): 40.00

NACA/Urea Co-crystal. A primary co-crystal screen was conducted where 28 potential co-crystal formers (CCFs) were screened (Table 18) in 6 solvent systems under 2 process relevant crystallization conditions, namely, thermal maturation and evaporation. A NACA/urea co-crystal was identified. The NACA/urea pattern 1 material was successfully scaled up from four solvents as a part of the secondary co-crystal screen, then fully characterized where it was found to be crystalline by XPRD and PLM with the expected XRPD pattern, thermally stable with high purity. NMR analysis confirmed an approximate stoichiometric content of urea contained within the material. The material appeared to be stable under ambient conditions and elevated temperature (80° C.) but unstable when stored at high humidity for prolonged periods, showing degradation to the diNACA. No signs of dissociation were identified in organic solvents and solvent/water mixtures with low water activity but was found to dissociate in deionized water and solvent/water mixtures with a high water activity. An additional DSC experiment with post-XRPD analysis confirmed that the exothermic event observed during the DSC cooling cycle is a recrystallization to NACA.

TABLE 18
Primary Co-Crystal Screen Co-Former List

| | Co-Former | GRAS* |
|---|---|---|
| 1 | 2-Picolinamide | Yes |
| 2 | 5-Methylfurfural | Yes |
| 3 | 5-Methylfurfurylamine | Yes |
| 4 | Adenine | Yes |
| 5 | Citric Acid | Yes |
| 6 | Glycine | Yes |
| 7 | Hippuric Acid | Yes |
| 8 | L-Aspartic Acid | Yes |
| 9 | L-Proline | Yes |
| 10 | L-Tyrosine | Yes |
| 11 | Malonic Acid | Yes |
| 12 | Melamine | Yes |
| 13 | Oxalic Acid | Yes |
| 14 | Theophylline | Yes |
| 15 | Tromethamine | Yes |
| 16 | Urea | Yes |
| 17 | Xanthine | Yes |
| 18 | 3,4-Dihydroxybenzoic Acid | Yes |
| 19 | Camphoric Acid | Yes |
| 20 | Cytosine | Yes |
| 21 | Formamide | Yes |
| 22 | L-Cysteine | Yes |
| 23 | L-Methionine | Yes |
| 24 | L-Serine | Yes |
| 25 | Threonine | Yes |
| 26 | DiNACA | Yes |
| 27 | N-Acetyl-L-cysteine | Yes |
| 28 | Succinic acid | Yes |

*GRAS: generally recognized as safe

NACA Sodium Salt. A salt screen was conducted using 6 solvent systems under 2 process-relevant crystallization conditions, namely thermal maturation and evaporation. From this study, one sodium salt was identified.

### Example 1: Isolation and Identification of Impurities B1 and B2

Step 3A filtrate, by the process of U.S. Pat. No. 10,590,073, was analyzed for trace component isolation and identification. Impurities B1 and B2 were isolated using Preparative HPLC Method (Table 19) and the isolated preparative fractions were analyzed using a mass spectrometric (MS)-compatible analytical HPLC Method for impurities B1 and B2 (Table 20):

TABLE 19
Preparative HPLC Method for Isolating Impurities B1 and B2

| | |
|---|---|
| Column | 19 × 50 mm, 5 μm Atlantis Prep T3 OBD by Waters (Milford, MA) |
| Mobile Phase A | 0.05% Trifluoroacetic Acid in Water |
| Mobile Phase B | Acetonitrile |
| Isocratic Method | Hold at 0% B for 4.0 minutes, 0-80% B over 0.1 minute, hold at 80% |
| | B for 0.9 minute, return to initial conditions |
| Flow Rate | 25 mL/min |
| Column Temperature | 25° C. |
| Wavelength | 214 nm |
| Sample Diluent | Water |

TABLE 20
MS-compatible Analytical HPLC Method for Impurities B1 and B2

| | |
|---|---|
| Column | 4.6 × 150 mm, 3.5 μm XSelect CSH C18 by Waters (Milford, MA) |
| Mobile Phase A | 0.05% Trifluoroacetic Acid in Water |
| Mobile Phase B | Acetonitrile |
| Gradient Method | Hold at 1% B for 1.0 minute, 0-10% B over 7.0 minutes, hold at |
| | 100% B for 3.0 minutes, return to initial conditions |
| Flow Rate | 1.0 mL/min |
| Column Temperature | 40° C. |
| Wavelength | 214 nm |
| Sample Diluent | Acetonitrile |

A representative preparative HPLC chromatogram, showing resolution between impurities B1 and B2, i.e., Fractions 1 and 2 (note: it is unknown which diastereomer is associated with which peak), is shown in FIG. 1. A total of 31 mg of Impurity 1 and 52 mg of enriched Impurity 2 were recovered. Impurity B1 was successfully isolated by pooling and drying the collected fraction yielding a single peak (FIG. 2). Likewise, Impurity B2 was successfully isolated by pooling and drying the collected fraction yielding a single peak (FIG. 3). Multiple preparative passes and variations to fraction collection were attempted. However, despite several attempts, isolation of Impurity B2 always resulted in a mixture of Impurities B1 and B2 by MS-compatible analytical HPLC.

Analytical peaks were evaluated by electrospray ionization mass spectrometry (FIGS. 4-8). The observed masses of 313.0941 [M+Na] and 291.1122 [M+H] for both Impurity B1 (Full ESI+ spectrum, FIG. 4; ESI+ MS/MS spectrum, FIG. 5; ESI+ MS/MS of 291.1 spectrum, FIG. 6) and Impurity B2 (Full scan ESI+ spectrum of Enriched B2, FIG. 7; ESI+ MS/MS of 313.1 spectrum of B2, FIG. 8) corresponds to a chemical formula C₁₀H₁₈N₄O₄S. The MS data shows key fragments that support the structure. The MS/MS fragment of 185.03577 corresponds to a chemical formula of C₅H₁₀N₂O₂S-Na and a fragment of C₅H₈N₂O₂-Na. A list of detected masses, their corresponding formulae, and gain and/or loss from the parent ion, are shown in Table 21.

TABLE 21
Observed masses, corresponding formula and gain/loss from parent mass.

| Observed Mass | Formula | Loss / Gain From Parent Mass |
|---|---|---|
| 313.09408 | C10H18N4O4SNa | +Na |
| 185.03555 | C5H10N2O2SNa | −C5H8N2O2/+Na |
| 151.04781 | C5H7N2O2Na | −C5H11N2O2S/ |
| | | +Na |
| 291.11217 | C10H19N4O4S | +H |
| 274.08559 | C10H16N3O4S | −NH2 |
| 257.05918 | C10H13N2O4S | −N2H5 |
| 229.0642 | C9H13N2O3S | −CH5N2O |
| 215.04856 | C8H11N2O3S | −C2H7N2O |
| 187.05364 | C7H11N2O2S | −C3H7N2O2 |
| 170.02706 | C7H8NO2S | −C3H10N3O2 |
| 163.0536 | C5H11N2O2S | −C5H7N2O2 |
| 146.027 | C5H8NO2S | −C5H10N3O2 |
| 118.03217 | C4H8NOS | −C4H10N3O |

Several Nuclear Magnetic Resonance (NMR) spectroscopy experiments were conducted to confirm structures of impurities B1 and B2: Correlated Spectroscopy (COSY; cross peaks in COSY are between protons that are coupled, i.e., what proton is coupled to what proton, and protons that are two, three, or sometimes four bonds apart may show cross peaks); Heteronuclear Multiple Bond Correlation spectroscopy (HMBC; correlates carbons and protons that are separated by two, three, and, sometimes in conjugated systems, four bonds, while direct one-bond correlations are suppressed, yielding connectivity information much like a proton-proton COSY); Heteronuclear Single Quantum Coherence spectroscopy (HSQC; determines proton-carbon single bond correlations, where the protons lie along the observed F2 (X) axis and the carbons are along the F1 (Y) axis); Nuclear Overhauser Effect spectroscopy (NOESY; correlation spectroscopy via transfer of magnetization among the spins through dipolar coupling, resembling diagonal and cross peaks of COSY and TOCSY); and Total Correlation spectroscopy (TOCSY; correlates all protons within a given spin system, not just between geminal or vicinal protons as in COSY, e.g., correlations are seen between distant protons as long as there are couplings between every intervening proton). The NMR data for Impurities B1 and B2 are consistent with each other and with diasteromeric structures (¹H-NMR Impurity B1, FIG. 9; ¹³C-NMR Impurity B1, FIG. 10; COSY NMR Impurity B1, FIG. 11; HMBC NMR Impurity B1, FIG. 12; HSQC NMR Impurity B11, FIG. 13; NOESY NMR Impurity B11, FIG. 14; TOCSY NMR Impurity B11, FIG. 15; ¹H-NMR Impurity B2, FIG. 16; ¹³C-NMR Impurity B21, FIG. 17; COSY NMR Impurity B2, FIG. 18; HMBC NMR Impurity B2, FIG. 19; HSQC NMR Impurity B2, FIG. 20; NOESY NMR Impurity B2, FIG. 21) shown in Table 22. In particular, the HMBC spectra show a strong correlation from the non-equivalent methylene protons to a carbon that is magnetically and chemically equivalent to the one to which they are directly attached. Correlation occurring through three bonds (and the heteroatom) for these molecules is consistent with dimers.

Diasteromeric structures for Impurities B1 and B2 are shown in Table 22. Absolute stereochemistry corresponding to each HPLC peak could not be determined using the data acquired. Therefore, peaks are typically assigned chronologically, numerically B1 and B2 as they elute on chromatogram but it is unknown which peak corresponds to which isomer.

TABLE 22
Properties of a diastereomeric pair of impurities, B1 and B1.

| | |
|---|---|
| 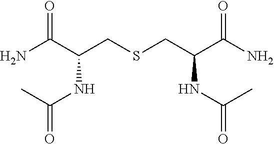 [SMILES: CC(=O)N[C@@H](CSC[C@H](NC(C)=O)C(N)=O)C(N)=O] | 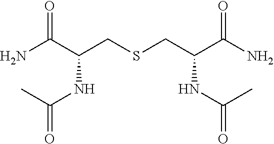 [SMILES: CC(=O)N[C@@H](CSC[C@@H](NC(C)=O)C(N)=O)C(N)=O] |
| (2R,2′R)-3,3′-thiobis(2- | (2R,2′S)-3,3′-thiobis(2- |
| acetamidopropanamide) | acetamidopropanamide) |
| Molecular formula: C₁₀H₁₈N₄O₄S | Molecular formula: C₁₀H₁₈N₄O₄S |
| Molecular weight: 290.34 g/mole | Molecular weight: 290.34 g/mole |
| No structural alert in silico. | No structural alert in silico. |

B1 and B2 are detected and resolved in HPLC Method II (US Patent 10590073) for purity in
NPI-001 at RRT 1.45 and 1.56. It is unknown which peak corresponds to which isomer.
Typical B1 levels in API: 0.08-0.41%
Typical B2 levels in API: 1.30-1.46%

### Example 2: Toxicological Qualification of Impurities B1 and B2

Non-clinical toxicology batch NPI-001 17-7352-01 was purposefully enriched in impurities B1, at 0.91%, and B2, at 2.27%, among others, for toxicological qualification of impurities under Good Laboratory Practices (GLPs).

Male and female rats (10 rats/sex/group) were dosed with vehicle, (0.05 M phosphate buffer, pH 7.0) or NPI-001 formulation (200, 600, or 2000 mg/kg) once daily by oral gavage at a dose volume of 10 mL/kg for at least 14 days, and euthanized one day following the last dose. Examined parameters included clinical observations, mortality and moribundity checks, body weights, food consumption, serum chemistry, hematology, coagulation, urinalysis, gross pathology, organ weights, and microscopic pathology.

After 14 days of once-daily oral administration of NPI-001 (200, 600, or 2000 mg/kg) in rats, none of the findings were considered to be adverse. Overall, NPI-001 was well tolerated at all dose levels tested. A No-Observed-Adverse-Effect Level (NOAEL) for NPI-001 API containing impurities B1, at 0.91%, and B2, at 2.27%, was determined to be 2000 mg/kg.

### Example 3: In Silico Assessment of Mutagenic Potential for Impurities B1 and B2

Impurities B1 and B2 were evaluated in silico for structural alerts associated with bacterial mutagenicity. To identify structural alerts, the molecules were analyzed using the Deductive Estimation of Risk from Existing Knowledge (Derek) and Leadscope Model Applier QSAR (Non-Human Genetic Toxicity module) software programs. Employing the two in silico predictive models (Derek and Leadscope) utilizes both expert systems and statistical models to assess structures, satisfying the ICH Draft Consensus Guideline M7 (Step 2): Assessment and Control of DNA Reactive (Mutagenic) Impurities in Pharmaceuticals to Limit Potential Carcinogenic Risk (Nacuity data on file). Therefore, B1 and B2 were found to be non-mutagenic based on results of in silico analyses.

### Example 4: Analysis of Impurities in NPI-001 API

Purified NPI-001 drug substance batches were manufactured according to the process in U.S. patent Ser. No. 10/590,073 and impurities B1 and B2 were quantitated by Method II (U.S. Pat. No. 10,590,073). Levels of impurities B1 and B2 in purified NPI-001 API batches are shown in Table 23.

TABLE 23
Levels of Impurities in NPI-001 API batches.

| | Impurities (%) | | | |
|---|---|---|---|---|
| Drug Substance | | | | Total Unspecified |
| Batch | diNACA* | Impurity B1 | Impurity B2 | Impurities |
| A | 0.83 | 0.41 | 1.30 | 0.20 |
| B | 0.87 | 0.35 | 1.46 | 0.12 |
| C | 1.5 | 0.08 | 1.30 | 0.24 |
| D | 1.5 | 0.09 | 1.32 | 0.24 |

*Post-recrystallization level with no chromatographic purification.

### Example 5: Analysis of Impurities in Non-Purified NPI-001 API

NPI-001 drug substance batch 15-02856-05 was synthesized by the process described in U.S. Pat. No. 9,763,9021B2 rather than the process of U.S. Pat. No. 10,590,073. Impurity analysis revealed the results listed in Table 24, showing (a) greater level of Total Unspecified Impurities and (b) no detectable levels of impurities B1 and B2. Total unspecified impurities in NACA synthesized according to U.S. Pat. No. 9,763,9021B2 was 1.55%, even after flash-chromatography purification.

TABLE 24
Levels of Impurities in NACA API batch 15-02856-05 According to
U.S. Pat. No. 9,763,902B2.

| | Impurities (%) | | | |
|---|---|---|---|---|
| | | Impurity | Impurity | Total Unspecified |
| NACA | diNACA | B1 | B2 | Impurities |
| Post- | *~4-5% | *NR | *NR | *NR |
| crystallization | | | | |
| before flash | | | | |
| chromatography | | | | |
| Nacuity batch 15- | 1.80 | <0.03 | <0.03 | 1.55 |
| 02856-05, Post- | | | | |
| flash | | | | |
| chromatography | | | | |

NR: Not Reported;
*U.S. Pat. No. 9,763,902B2;
**Nacuity data on file.

### Example 6: Analysis of Impurities B and B2 in NPI-001 Tablets

NPI-001 Tablets were manufactured as described in U.S. patent Ser. No. 10/590,073. Impurities B1 and B2 were quantitated in NPI-001 Tablets (FIG. 22) utilizing an HPLC Method described in Table 25 with the levels shown in Table 26. A chromatogram of Placebo, showing no impurities B1 and B2 (nor NPI-001), is shown in FIG. 23. Results in Table 26 demonstrate that Impurities B1 and B2 do not significantly change at long-term storage conditions, indicative that these impurities are synthetic by-products rather than degradation products.

TABLE 25
Analytical HPLC Method CTMLP-4248 for B1 and B2 in NPI-001 Tablets.

| Column | Zorbax SB-Aq StableBond HPLC column, 4.6 mm × 250 mm, 5 μm | | |
|---|---|---|---|
| Mobile Phase A | 0.02% H₃PO₄ | | |
| Mobile Phase B | Acetonitrile | | |
| Gradient Method | Time (minutes) | % A | % B |
| | 0.0 | 100 | 0 |
| | 5.0 | 100 | 0 |
| | 20.0 | 0 | 100 |
| | 25.0 | 0 | 100 |
| | 25.01 | 100 | 0 |
| | 30.0 | 100 | 0 |

| | |
|---|---|
| Flow Rate | 1.0 mL/min |
| Column Temperature | 40° C. |
| Wavelength | 214 nm |
| Sample Diluent | Mobile phase A |

TABLE 26
Levels of Impurities B1 and B1 in NPI-001 Tablets

| | Batch* (analysis date) | | | |
|---|---|---|---|---|
| | Batch “ZK” | Batch “ZK” | Batch “YH” | Batch “YH” |
| Im- | (stability | (after storage at | (stability | (after storage at |
| pur- | protocol | 30°C/65% RH × | protocol | 30°C/65% RH × |
| ity | initiation) | 20 months) | initiation) | 20 months) |
| B1 | 0.31% | 0.33% | 0.14% | 0.14% |
| B2 | 1.1% | 1.2% | 1.1% | 1.3% |

*Manufactured using NPI-001 API batch C (Table 23)

### Example 7: Analysis of Impurities in NPI-002 API

Purified NPI-002 (diNACA) drug substance batches were manufactured according to the process in U.S. patent Ser. No. 10/590,073 and impurities B1 and B2 were quantitated by Method “1417” (Table 28). Levels of impurities B1 and B2 in purified NPI-002 API batches are shown in Table 27.

TABLE 27
Levels of Impurities in NPI-002 API batches

| | Individual Specified Impurities (%)* | |
|---|---|---|
| Drug Substance Batch | Impurity B1 | Impurity B2 |
| “03201” | 0.08 | 0.21 |

*Post-recrystallization level with no chromatographic purification

TABLE 28
Analytical HPLC Method “1414” for NPI-002 API.

| Column | Phenomenex Synergy Hydro-RP HPLC column, | | |
|---|---|---|---|
| | 4 μm, 4.6 × 250 mm | | |
| Mobile Phase A | 0.02% H₃PO₄ | | |
| Mobile Phase B | Acetonitrile | | |
| Gradient Method | Time (minutes) | % A | % B |
| | 0.0 | 98 | 2 |
| | 5.0 | 98 | 2 |
| | 20.0 | 90 | 10 |
| | 35.0 | 0 | 100 |
| | 40 | 0 | 100 |
| | 40.01 | 98 | 2 |
| | 45 | 98 | 2 |

| | |
|---|---|
| Flow Rate | 1.0 mL/min |
| Column Temperature | 30° C. |
| Wavelength | 214 nm |
| Sample Diluent | Mobile phase A |

The manufacturing process of U.S. Pat. No. 10,590,073 yields N-Acetylcysteine amide (NACA, NPI-001) with greater purity, i.e., NACA containing diNACA at 0.83 to 1.50% and total unspecified impurities ≤0.24%, without the need for chromatographic purification. NACA synthesized by other reported process, e.g., the process described in U.S. Pat. No. 9,763,902 typically contains diNACA at 4-5%, requiring choroatographic purification. Even after chromatographic purification, NACA prepared buy U.S. Pat. No. 9,763,902 typically contains diNACA at 1.8% and total unspecified impurities at 1.55%. Hence, N-Acetylcysteine amide (NACA, NPI-001) manufactured according to the process of U.S. Pat. No. 10,590,073 is considered purified N-Acetylcysteine amide, purified NACA or purified NPI-001.

Purified N-Acetylcysteine amide, purified NACA or purified NPI-001 manufactured according to the process of U.S. Pat. No. 10,590,073 can be confirmed by the presence of innocuous impurities B1 and B2 whereas non-purified NPI-001 manufactured by a process other than the process of U.S. Pat. No. 10,590,073 (e.g., according to U.S. Pat. No. 9,763,902) does not contain detectable levels of impurities B1 and B2. Therefore, the presence of impurities B1 and B2 indicates N-Acetylcysteine amide, NACA or NPI-001 synthesized according to the process of U.S. Pat. No. 10,590,073 rather than by some other method. The presence or absence of Impurities B1 and B2 serves as an anticounterfeit marker for Purified N-Acetylcysteine amide, purified NACA or purified NPI-001. Analysis of N-Acetylcysteine amide, NACA or NPI-001 API, or N-Acetylcysteine amide, NACA or NPI-001 Tablets, can be conducted according to HPLC methods described herein. The presence of impurities B1 and B2 indicates that N-Acetylcysteine amide, NACA or NPI-001 API, or N-Acetylcysteine amide, NACA or NPI-001 Tablets, were manufactured according to the process of U.S. Pat. No. 10,590,073. The absence of impurities B1 and B2 indicates that N-Acetylcysteine amide API or N-Acetylcysteine amide Tablets were manufactured according a process other than the process of U.S. Pat. No. 10,590,073.

Further, the manufacturing process of U.S. Pat. No. 10,590,073 yields diNACA (NPI-002) which can be can be confirmed by the presence of innocuous impurities B1 and B2. Therefore, the presence of impurities B1 and B2 indicates diNACA (NPI-002) synthesized according to the process of U.S. Pat. No. 10,590,073 rather than by some other method. It is contemplated that any embodiment discussed in this specification can be implemented with respect to any method, kit, reagent, or composition of the disclosure, and vice versa. Furthermore, compositions of the disclosure can be used to achieve methods of the disclosure.

### Embodiments

Embodiment 1. A drug substance used in a method for treating a disease or condition in an animal or human in need thereof, the method comprising:
identifying that a subject has a disease or condition caused by oxidative stress;
providing an effective amount of an N-acetylcysteine amide (NACA) sufficient to increase a concentration of NACA; and
treating the disease or condition with NACA that comprises at least one of impurities B1 or B2, which are indicative of NACA manufactured by a process comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base.

Embodiment 2. The method of embodiment 1, wherein the NACA comprises impurities B1 and B2 at any detectable concentration as determined by HPLC analysis.

Embodiment 3. The method of embodiments 1 or 2, wherein the NACA comprises at least one of:
impurities B1 and B2 at levels greater than zero but B1 less than 0.91% peak area percents (PA %) and B2 less than 2.27 PA %, as determined by HPLC peak area analysis;
impurities B1 and B2 at levels greater than zero but B1 less than about 2 PA % and B2 less than about 4 PA %, as determined by HPLC peak area analysis;
impurities B1 and B2 at levels greater than zero but B1 less than 5% and B2 less than 7 PA %, as determined by HPLC peak area analysis; or
impurities B1 at about 0.05 PA % to 1.0 PA % and B2 at about 1.0 to 2.0 PA %, as anticounterfeit markers.

Embodiment 4. The method of any one of embodiments 1 to 3, wherein the NACA comprises impurities B1 and B2 at levels that have been toxicologically qualified and serve as markers for NACA.

Embodiment 5. The method of any one of embodiments 1 to 4, wherein the NACA is provided orally, peritoneally, intravenously, dermally, bucally, sublingually, topically, topical ocularly, intraocularly, intravitreally, transmucosally, intradermally, subcutaneously, or pulmonarily.

Embodiment 6. The method of any one of embodiments 1 to 5, wherein the NACA is administered in the form of a gel, ointment, liniment, lotion, capsule, cream, implant, minitablet, pill, powder, suspension, tablet, emulsion or a suppository.

Embodiment 7. The method of any one of embodiments 1 to 6, wherein the disease is retinitis pigmentosa, retinitis pigmentosa associated with Usher syndrome, age-related macular degeneration, cystinosis, corneal endothelial loss, amyotrophic lateral sclerosis (ALS), Alzheimer's disease, Parkinson's disease, Huntington's disease, a liposomal storage disease, a gain of function mutation in ACOX1, or Hereditary cystatin C amyloid angiopathy (HCCAA).

Embodiment 8. The method of any one of embodiments 1 to 7, wherein the condition is traumatic brain injury, cataract formation, binge eating, methamphetamine abuse, alcohol abuse, or acetaminophen overdose.

Embodiment 9. The method of any one of embodiments 1 to 8, wherein the NACA is administered topically in the form of a gel, an ointment, a liniment, a lotion, a cream, a pill, a powder, a solution, a suspension, an emulsion, an implant, a sublingual formulation or a suppository.

Embodiment 10. The method of any one of embodiments 1 to 9, wherein the NACA is administered topically in the form of a solution that is formed by mixing lyophilized NACA with diluent prior to administration.

Embodiment 11. The method of any one of embodiments 1 to 10, wherein the NACA is administered by an intradermal, intramuscular, intraocular, intravitreal or subcutaneous injection.

Embodiment 12. The method of any one of embodiments 1 to 11, wherein a dose of NACA is between 1 and 10 mg/day, between 10 and 200 mg/day, between 60 and 80 mg/day, between 250 and 500 mg/day, between 501 and 1,500 mg/day, or between 1,501 and 4,000 mg/day.

Embodiment 13. The method of any one of embodiments 1 to 12, wherein the NACA is dosed daily for over several days, 1 week, 1 month, 6 months, 1 year or 5 years or longer.

Embodiment 14. The method of any one of embodiments 1 to 13, wherein the NACA is purified as a result of crystallization, needing no further purification by at least one of: column chromatography, paper chromatography, thin layer chromatography, high performance liquid chromatography, fast liquid chromatography, supercritical fluid chromatography, affinity chromatography, reversed phase chromatography, two dimensional chromatography, counter current chromatography, or flash chromatography.

Embodiment 15. The method of any one of embodiments 1 to 14, wherein the NACA contains less than 2 PA % diNACA.

Embodiment 16. The method of any one of embodiments 1 to 15, wherein the NACA contains less than 4 PA % diNACA.

Embodiment 17. A pharmaceutical composition used for treating diseases and conditions in an animal or human comprising:
an N-acetylcysteine amide (NACA) that comprises impurities B1 and B2 that are indicative of NACA manufactured by:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base; and
a pharmaceutically acceptable carrier.

Embodiment 18. The pharmaceutical of embodiment 17, wherein the NACA is a tablet is dosed once daily, dosed twice daily, or dosed more than twice daily.

Embodiment 19. The pharmaceutical of embodiments 17 or 18, wherein the NACA contains impurities B1 and B2 at any detectable concentration as determined by HPLC analysis.

Embodiment 20. The pharmaceutical of any one of embodiments 17 to 19, wherein the NACA is not purified by chromatography.

Embodiment 21. A chemical mixture of di-N-acetylcysteine amide (diNACA, NPI-001) comprising the following chemical structures:

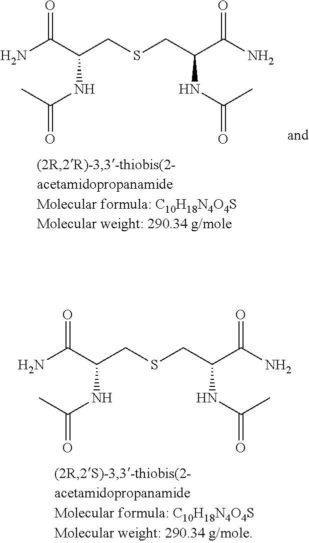 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]

wherein the diNACA contains total unspecified impurities of less than 1.55 peak area percents (PA %) based on HPLC peak area.

Embodiment 22. The mixture of any one of embodiments 21, wherein total unspecified impurities at about ≤0.24 PA %, less than about 0.5 PA %, less than about 1 PA %, less than about 1.55 PA %.

Embodiment 23. The mixture of embodiments 21 or 22, wherein the mixture contains less than 2 PA % diNACA, or less than 4 PA % diNACA.

Embodiment 24. The mixture of any one of embodiments 21 to 23, wherein the mixture is a purified NACA.

Embodiment 25. Chemical moieties comprising the following chemical structures:

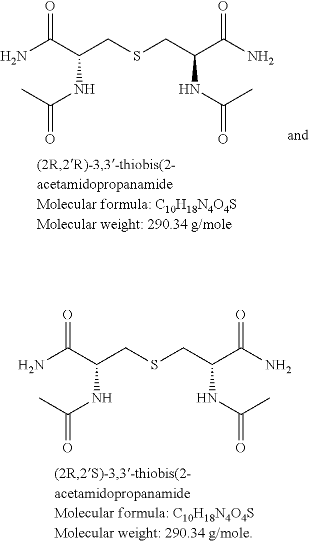 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]

Embodiment 26. A NACA drug substance or drug product prepared by a method comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base, wherein the drug substance or drug product comprises at least one of:
impurity B1 as an identifier, indicator, marker, or anticounterfeit measure; or
impurity B2 as an identifier, indicator, marker, or anticounterfeit measure.

Embodiment 27. The NACA prepared by method of embodiment 26, wherein the drug substance or drug product comprises at least one of: about 0.05 to 0.5% to 1% impurity B1; or less than 5% impurity B1; about 0.05% to 1.5% to 2% impurity B2; or less than 5% impurity B2.

Embodiment 28. The NACA prepared by method of embodiments 26 or 27, wherein the presence of at least one of impurity B1 or impurity B2 comprises an anticounterfeit marker, identifier or indicator of the synthetic process.

Embodiment 29. A di-N-acetylcysteine amide (diNACA) prepared by a method comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester; and
mixing the di-N-acetylcystine dimethylester with ammonium hydroxide to form di-N-acetylcystine amide, wherein the diNACA drug substance or diNACA drug product contains at least one of:
impurity B1 as an identifier, indicator, marker, or anticounterfeit measure; or
impurity B2 as an identifier, indicator, marker, or anticounterfeit measure.

Embodiment 30. The diNACA prepared by method of embodiment 29, wherein the diNACA drug substance or diNACA drug product contains at least one of: about 0.05 to 0.5% to 1% impurity B1; or less than 5% impurity B1; about 0.05% to 1.5% to 2% impurity B2; or less than 5% impurity B2.

Embodiment 31. The diNACA prepared by method of embodiments 29 or 30, wherein the presence of at least one of impurity B1 or impurity B2 comprises an anticounterfeit marker, identifier or indicator of the synthetic process.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “of” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A drug substance used in a method for treating a disease or condition in an animal or human in need thereof, the method comprising:
identifying that a subject has a disease or condition caused by oxidative stress;
providing an effective amount of an N-acetylcysteine amide (NACA) sufficient to increase a concentration of NACA; and
treating the disease or condition with NACA that comprises at least one of impurities B1 or B2, which are indicative of NACA manufactured by a process comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base.

2. The method of claim 1, wherein the NACA comprises impurities B1 and B2 at any detectable concentration as determined by HPLC analysis.

3. The method of claim 1, wherein the NACA comprises at least one of:
impurities B1 and B2 at levels greater than zero but B1 less than 0.91% peak area percents (PA %) and B2 less than 2.27 PA %, as determined by HPLC peak area analysis;
impurities B1 and B2 at levels greater than zero but B1 less than about 2 PA % and B2 less than about 4 PA %, as determined by HPLC peak area analysis;
impurities B1 and B2 at levels greater than zero but B1 less than 5% and B2 less than 7 PA %, as determined by HPLC peak area analysis; or
impurities B1 at about 0.05 PA % to 1.0 PA % and B2 at about 1.0 to 2.0 PA %, as anticounterfeit markers.

4. The method of claim 1, wherein the NACA comprises impurities B1 and B2 at levels that have been toxicologically qualified and serve as markers for NACA.

5. The method of claim 1, wherein the NACA is provided orally, peritoneally, intravenously, dermally, bucally, sublingually, topically, topical ocularly, intraocularly, intravitreally, transmucosally, intradermally, subcutaneously, or pulmonarily.

6. The method of claim 1, wherein the NACA is administered in the form of a gel, ointment, liniment, lotion, capsule, cream, implant, minitablet, pill, powder, suspension, tablet, emulsion or a suppository.

7. The method of claim 1, wherein the disease is retinitis pigmentosa, retinitis pigmentosa associated with Usher syndrome, age-related macular degeneration, cystinosis, corneal endothelial loss, amyotrophic lateral sclerosis (ALS), Alzheimer's disease, Parkinson's disease, Huntington's disease, a liposomal storage disease, a gain of function mutation in ACOX1, or Hereditary cystatin C amyloid angiopathy (HCCAA).

8. The method of claim 1, wherein the condition is traumatic brain injury, cataract formation, binge eating, methamphetamine abuse, alcohol abuse, or acetaminophen overdose.

9. The method of claim 1, wherein the NACA is administered topically in the form of a gel, an ointment, a liniment, a lotion, a cream, a pill, a powder, a solution, a suspension, an emulsion, an implant, a sublingual formulation or a suppository.

10. The method of claim 1, wherein the NACA is administered topically in the form of a solution that is formed by mixing lyophilized NACA with diluent prior to administration.

11. The method of claim 1, wherein the NACA is administered by an intradermal, intramuscular, intraocular, intravitreal or subcutaneous injection.

12. The method of claim 1, wherein a dose of NACA is between 1 and 10 mg/day, between 10 and 200 mg/day, between 60 and 80 mg/day, between 250 and 500 mg/day, between 501 and 1,500 mg/day, or between 1,501 and 4,000 mg/day.

13. The method of claim 1, wherein the NACA is dosed daily for over several days, 1 week, 1 month, 6 months, 1 year or 5 years or longer.

14. The method of claim 1, wherein the NACA is purified as a result of crystallization, needing no further purification by at least one of: column chromatography, paper chromatography, thin layer chromatography, high performance liquid chromatography, fast liquid chromatography, supercritical fluid chromatography, affinity chromatography, reversed phase chromatography, two dimensional chromatography, counter current chromatography, or flash chromatography.

15. The method of claim 1, wherein the NACA contains less than 2 PA % diNACA.

16. The method of claim 1, wherein the NACA contains less than 4 PA % diNACA.

17. A pharmaceutical composition used for treating diseases and conditions in an animal or human comprising:
an N-acetylcysteine amide (NACA) that comprises impurities B1 and B2 that are indicative of NACA manufactured by:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base; and
a pharmaceutically acceptable carrier.

18. The pharmaceutical of claim 17, wherein the NACA is a tablet is dosed once daily, dosed twice daily, or dosed more than twice daily.

19. The pharmaceutical of claim 17, wherein the NACA contains impurities B1 and B2 at any detectable concentration as determined by HPLC analysis.

20. The pharmaceutical of claim 17, wherein the NACA is not purified by chromatography.

21. A chemical mixture of di-N-acetylcysteine amide (diNACA, NPI-001) comprising the following chemical structures: 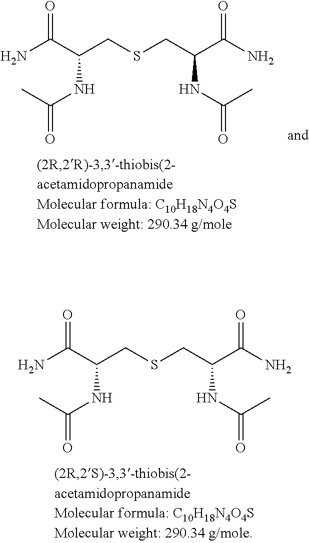 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]
wherein the diNACA contains total unspecified impurities of less than 1.55 peak area percents (PA %) based on HPLC peak area.

22. The mixture of claim 21, wherein total unspecified impurities at about ≤0.24 PA %, less than about 0.5 PA %, less than about 1 PA %, less than about 1.55 PA %.

23. The mixture of claim 21, wherein the mixture contains less than 2 PA % diNACA, or less than 4 PA % diNACA.

24. The mixture of claim 21, wherein the mixture is a purified NACA.

25. Chemical moieties comprising the following chemical structures:

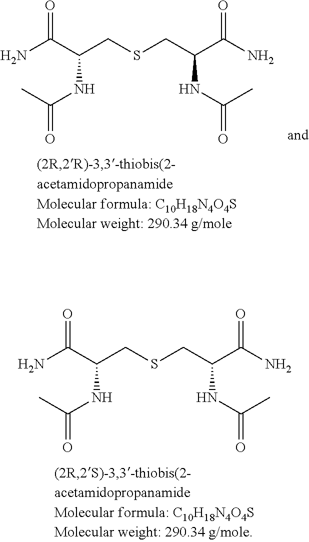 [SMILES: CC(=O)N[C@@H](CSC[C@H](NC(C)=O)C(N)=O)C(N)=O ; CC(=O)N[C@@H](CSC[C@@H](NC(C)=O)C(N)=O)C(N)=O]

26. A NACA drug substance or drug product prepared by a method comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester;
mixing the di-N-acetylcystine dimethylester with ammonium hydroxide to form di-N-acetylcystine amide; and
reducing the di-N-acetylcystine amide to NACA with a reducing agent, an organic solvent, and a base, wherein the drug substance or drug product comprises at least one of:
impurity B1 as an identifier, indicator, marker, or anticounterfeit measure; or
impurity B2 as an identifier, indicator, marker, or anticounterfeit measure.

27. The NACA prepared by method of claim 26, wherein the drug substance or drug product comprises at least one of: about 0.05 to 0.5% to 1% impurity B1; or less than 5% impurity B1; about 0.05% to 1.5% to 2% impurity B2; or less than 5% impurity B2.

28. The NACA prepared by method of claim 26, wherein the presence of at least one of impurity B1 or impurity B2 comprises an anticounterfeit marker, identifier or indicator of the synthetic process.

29. A di-N-acetylcysteine amide (diNACA) prepared by a method comprising:
contacting cystine with methanol and a chlorinating reagent to form an organic solution containing cystine dimethylester dihydrochloride and optionally isolating and drying the cystine dimethylester dihydrochloride;
combining the dried or undried cystine dimethylester dihydrochloride with triethylamine, acetic anhydride, and acetonitrile to form di-N-acetylcystine dimethylester; and
mixing the di-N-acetylcystine dimethylester with ammonium hydroxide to form di-N-acetylcystine amide, wherein a diNACA drug substance or diNACA drug product contains at least one of:
impurity B1 as an identifier, indicator, marker, or anticounterfeit measure; or
impurity B2 as an identifier, indicator, marker, or anticounterfeit measure.

30. The diNACA prepared by method of claim 29, wherein the diNACA drug substance or diNACA drug product contains at least one of: about 0.05 to 0.5% to 1% impurity B1; or less than 5% impurity B1; about 0.05% to 1.5% to 2% impurity B2; or less than 5% impurity B2.

31. The diNACA prepared by method of claim 29, wherein the presence of at least one of impurity B1 or impurity B2 comprises an anticounterfeit marker, identifier or indicator of the synthetic process.
